(19) United States
(12) Patent Application Publication
(10) Pub. No.: US 2012/0041171 A1
(43) Pub. Date: Feb. 16, 2012

(54) PROCESS FOR THE PRODUCTION OF A RECOMBINANT POLYPEPTIDE OF INTEREST

(75) Inventors: Noel Nicolaas Maria Elisabeth Van Peij, Delfgauw (NL); Johannes Andries Roubos, Pijnacker (NL); Hein Stam, Huizen (NL); Peter Jozef Ida Van De Vondervoort, Schipluiden (NL)

(73) Assignee: DSM IP ASSETS B.V., Heerlen (NL)

(21) Appl. No.: 13/265,303

(22) PCT Filed: Apr. 14, 2010
(86) PCT No.: PCT/EP2010/054890

§ 371 Date: Oct. 19, 2011

(30) Foreign Application Priority Data
Apr. 22, 2009 (EP) 09158538.0
Apr. 22, 2009 (EP) 09158539.8
Apr. 22, 2009 (EP) 09158540.6
Apr. 22, 2009 (EP) 09158541.4
Apr. 22, 2009 (EP) 09158542.2

(51) Int. Cl. C07K 19/00; C12N 15/62; C12N 15/63; C12P 21/00; C07K 4/00; C07K 14/00; C12N 15/11; C12P 21/02; C12N 1/15

(52) U.S. Cl. 530/300; 435/69.7; 530/350; 536/23.4; 435/320.1; 435/254.11; 536/23.1

(57) ABSTRACT

The present invention relates to a process for the production of a recombinant polypeptide of interest, a polypeptide obtained by said process, a recombinant polynucleotide, an expression vector, an expression construct and to the use of a specific signal peptide and of a polynucleotide encoding said specific signal peptide for the production of a recombinant polypeptide of interest.

### FIELD OF THE INVENTION

The present invention relates to a process for the production of a recombinant polypeptide of interest, a polypeptide obtained by said process, a recombinant polynucleotide, an expression vector, an expression construct and to the use of a specific signal peptide and of a polynucleotide encoding said specific signal peptide for the production of a recombinant polypeptide of interest.

### BACKGROUND OF THE INVENTION

The production of recombinant polypeptides in filamentous fungal host cells is known in the art. Current production of polypeptides is performed in various ways.

The state of the art process for the production of recombinant polypeptides is by means of fermentation of a host cell comprising an expression construct, said expression construct comprising inter alia a promoter operably linked to a polynucleotide encoding the polypeptide of interest. To direct the polypeptide of interest to the secretory pathway of the host cell, the polypeptide of interest comprises a signal sequence. In Broekhuijsen et al (Journal of Biotechnology, 31 (1993) 135-145, Broekhuijsen et al; Secretion of heterologous proteins by Aspergillus niger: Production of active human interleukin-6 in a protease deficient mutant by KEX2-like processing of a glucoamylase-hIL6 fusion protein), a recombinant protein is expressed in Aspergillus niger using the signal sequence of the secreted polypeptide glucoamylase.

In an industrial context, high yields of polypeptides produced are required.

The yield of production of the recombinant polypeptide of interest may be enhanced by increasing the secretion efficiency.

Consequently, to enhance the yield of production of a polypeptide of interest, there is a need to improve secretion efficiency.

It is an object of the invention to provide an improved process for the production of a recombinant polypeptide.

### DESCRIPTION OF THE FIGURES

FIG. 1 depicts a plasmid map of expression vector pGBFINFUA-1 (described in WO2008/000632). pGBFINFUA-1 is also representative for plasmid pGBFINFUA-3 and pGBFINFUA-21. Indicated are the glaA flanking regions relative to the sequences of the amyB promoter and the A. niger amyB cDNA sequence encoding alpha-amylase with variant signal sequences introduced. The E. coli DNA can be removed by digestion with restriction enzyme NotI, prior to transformation of the A. niger strains.

FIG. 2 depicts a plasmid map of expression vector pGBFINFUA-6 (construction described in Example 1). pGBFINFUA-6 is also representative for plasmid pGBFINFUA-8, pGBFINFUA-11, pGBFINFUA-12, pGBFINFUA-13, pGBFINFUA-15, pGBFINFUA-16 and pGBFINFUA-18. Indicated are the glaA flanking regions relative to the sequences of the glaA promoter and the A. niger amyB cDNA sequence encoding alpha-amylase with variant signal sequences introduced. The E. coli DNA can be removed by digestion with restriction enzyme NotI, prior to transformation of the A. niger strains.

FIG. 3 depicts a schematic representation of integration through single homologous recombination. The expression vector comprises the selectable amdS marker, and a promoter connected to the amyB gene, which contains variant signal sequences. These features are flanked by homologous regions of the glaA locus (3′ glaA and 3″ glaA, respectively) to direct integration at the genomic glaA locus.

FIG. 4 depicts alpha-amylase activity in culture broth of A. niger strains expressing the different amyB constructs, all under control of the glaA promoter. Depicted is the alpha-amylase activity in culture broth of A. niger strains expressing an amyB construct, wherein signal sequences have been modified in the different constructs. Details about the different constructs can be found in Table 1. Alpha-amylase activities are depicted in relative alpha-amylase units [AU], with the average of the FUA-6 one-copy strain of the FUA6 group of 3 strains at day 3 set at 100%. For all transformant groups indicated, three transformants were isolated and cultivated independently.

FIG. 5 depicts alpha-amylase activity in culture broth of A. niger strains expressing two different amyB constructs, both under control of the amyB promoter. Depicted is the alpha-amylase activity in culture broth of A. niger strains expressing a native amyB construct (pGBFINFUA-3), wherein the amyB signal sequence was modified into a codon optimized pmeA signal sequence (pGBFINFUA-21), according a method of the invention. Details about the two constructs can be found in Table 2. Alpha-amylase activities are depicted in relative alpha-amylase units [AU], with the average of the FUA-3-1 one-copy strain of the FUA3 group of 3 strains at day 3 set at 100%. For the two transformant groups indicated, three transformants were isolated and cultivated independently.

FIG. 6 depicts glucose oxidase activity in culture broth of A. niger strains expressing two different constructs encoding P. chrysogenum glucose oxidase GoxA, both under control of the glaA promoter. Depicted is glucose oxidase activity in culture broth of A. niger strains expressing a native goxA construct (GOX-1-#), wherein the codon optimised goxA signal sequence was modified into a codon optimised pmeA signal sequence (GOX-2-#), according a method of the invention. Glucose oxidase activities are depicted in relative glucose oxidase units [AU]. For the two transformant groups indicated, five transformants were isolated and cultivated independently.

### DETAILED DESCRIPTION OF THE INVENTION

Surprisingly, it has been established that production of a recombinant polypeptide of interest can be improved by the use of specific signal sequences. Accordingly, in a first aspect of the present invention there is provided a process for the production of a recombinant polypeptide of interest comprising:
(i) cultivation of a filamentous fungal host cell under conditions conducive to the production of said polypeptide, said filamentous fungal host cell comprising a first polynucleotide linked in translational reading frame to a second polynucleotide, said second polynucleotide encoding a polypeptide of interest, said first polynucleotide encoding a signal peptide selected from the group consisting of:
   a) SEQ ID NO: 25,
   b) a variant of SEQ ID NO: 25 of between 15 and 23 amino acids, wherein at least 8 amino acids are identical to the first 10 amino acids of SEQ ID NO: 25 at corresponding positions,
   c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   d) SEQ ID NO: 39,
   e) a variant of SEQ ID NO: 39 of between 15 and 23 amino acids, wherein at least 8 amino acids are identical to the first 10 amino acids of SEQ ID NO: 39 at corresponding positions,
   f) a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   g) SEQ ID NO: 44,
   h) a variant of SEQ ID NO: 44 of between 15 and 23 amino acids, wherein at least 8 amino acids are identical to the first 10 amino acids of SEQ ID NO: 44 at corresponding positions,
   i) a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   j) SEQ ID NO: 34,
   k) a variant of SEQ ID NO: 34 of between 15 and 23 amino acids, wherein at least 8 amino acids are identical to the first 10 amino acids of SEQ ID NO: 34 at corresponding positions,
   l) a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
(ii) and optionally, isolation of said polypeptide from the culture medium.

The process described here above is herein referred to as the process according to the invention.

According to an embodiment, in the process of the invention, the signal peptide is SEQ ID NO: 25.

According to another embodiment, in the process of the invention, the signal peptide is a variant of SEQ ID NO: 25 of between 15 and 23 amino acids, wherein at least 8 amino acids are identical to the first 10 amino acids of SEQ ID NO: 25 at corresponding positions.

According to another embodiment, in the process of the invention, the signal peptide is a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, in the process of the invention, the signal peptide is SEQ ID NO: 39.

According to another embodiment, in the process of the invention, the signal peptide is a variant of SEQ ID NO: 39 of between 15 and 23 amino acids, wherein at least 8 amino acids are identical to the first 10 amino acids of SEQ ID NO: 39 at corresponding positions.

According to another embodiment, in the process of the invention, the signal peptide is a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, in the process of the invention, the signal peptide is SEQ ID NO: 44.

According to another embodiment, in the process of the invention, the signal peptide is a variant of SEQ ID NO: 44 of between 15 and 23 amino acids, wherein at least 8 amino acids are identical to the first 10 amino acids of SEQ ID NO: 44 at corresponding positions.

According to another embodiment, in the process of the invention, the signal peptide is a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, in the process of the invention, the signal peptide is SEQ ID NO: 34.

According to another embodiment, in the process of the invention, the signal peptide is a variant of SEQ ID NO: 34 of between 15 and 23 amino acids, wherein at least 8 amino acids are identical to the first 10 amino acids of SEQ ID NO: 34 at corresponding positions.

According to another embodiment, in the process of the invention, the signal peptide is a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

Preferably, in the process according to the invention, when the signal peptide is
(a): SEQ ID NO: 25,
(b): a variant of SEQ ID NO: 25 of between 15 and 23 amino acids, wherein at least 8 amino acids are identical to the first 10 amino acids of SEQ ID NO: 25 at corresponding positions, or
(c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   the polypeptide of interest is not a pectin methyl esterase, more preferably the polypeptide of interest is not a pectin methyl esterase from Erwinia chrysanthemi. More preferably, when the signal peptide is (b) or (c), the polypeptide of interest is not a pectin methyl esterase, even more preferably the polypeptide of interest is not a pectin methyl esterase from Erwinia chrysanthemi.

Preferably, the first polynucleotide when encoding SEQ ID NO: 25, is a polynucleotide according to SEQ ID NO: 29. Preferably, the first polynucleotide when encoding SEQ ID NO: 39, is a polynucleotide according to SEQ ID NO: 38. Preferably, the first polynucleotide when encoding SEQ ID NO: 44, is a polynucleotide according to SEQ ID NO: 43. Preferably, the first polynucleotide when encoding SEQ ID NO: 34, is a polynucleotide according to SEQ ID NO: 33.

Preferably, the variant of SEQ ID NO: 25 of between 15 and 23 amino acids, wherein at least 8 amino acids are identical to the first 10 amino acids of SEQ ID NO: 25 at corresponding positions, is a variant wherein at least 9 amino acids are identical to the first 10 amino acids of SEQ ID NO: 25 at corresponding positions. More preferably, 10 amino acids are identical to the first 10 amino acids of SEQ ID NO: 25 at corresponding positions.

Preferably, the variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu,
c) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu, and wherein the amino acids of the last three positions of the variant are Ala, Leu and Ala.

Preferably, the variant of SEQ ID NO: 39 of between 15 and 23 amino acids, wherein at least 8 amino acids are identical to the first 10 amino acids of SEQ ID NO: 39 at corresponding positions, is a variant wherein at least 9 amino acids are identical to the first 10 amino acids of SEQ ID NO: 39 at corresponding positions. More preferably, 10 amino acids are identical to the first 10 amino acids of SEQ ID NO: 39 at corresponding positions.

Preferably, the variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 39 of between 15 to 23 amino acids, wherein the amino acid at position 1 is Met, the amino acid at position 2, 3 and/or 5 is Val or Lys and wherein a contiguous stretch of 10 amino acids comprises at least 5 amino acids selected from Ala or Leu,
b) a variant of SEQ ID NO: 39 of between 15 to 23 amino acids, wherein the amino acid at position 1 is Met, the amino acid at position 2 is Val and the amino acid at position 5 is Lys and wherein a contiguous stretch of 10 amino acids comprises at least 5 amino acids selected from Ala or Leu.

Preferably, the variant of SEQ ID NO: 44 of between 15 and 23 amino acids, wherein at least 8 amino acids are identical to the first 10 amino acids of SEQ ID NO: 44 at corresponding positions, is a variant wherein at least 9 amino acids are identical to the first 10 amino acids of SEQ ID NO: 44 at corresponding positions. More preferably, 10 amino acids are identical to the first 10 amino acids of SEQ ID NO: 44 at corresponding positions.

Preferably, the variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 44 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 44 of between 15 to 23 amino acids, wherein the amino acid at position 1 is Met, the amino acid at position 2 is Val and the amino acid at position 4 is Lys and wherein a contiguous stretch of 10 amino acids comprises at least 5 amino acids selected from Ala or Leu.

Preferably, the variant of SEQ ID NO: 34 of between 15 and 23 amino acids, wherein at least 8 amino acids are identical to the first 10 amino acids of SEQ ID NO: 34 at corresponding positions, is a variant wherein at least 9 amino acids are identical to the first 10 amino acids of SEQ ID NO: 34 at corresponding positions. More preferably, 10 amino acids are identical to the first 10 amino acids of SEQ ID NO: 34 at corresponding positions.

Preferably, the variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu,
b) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu,
c) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu, and wherein the amino acids of the last three positions of the variant are Ala, Leu and Ala,
e) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu,
f) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids of the last three positions of the variant are Ala, Leu and Ala,
g) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids at the three last positions of the variant are Ala, Leu, Ala,
h) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Ala,
i) a variant of SEQ ID NO: 34 of between 15 to 23 amino acids, wherein the amino acid at position 1 is Met, the amino acid at position 2 is Val and wherein a contiguous stretch of 10 amino acids comprises at least 4 Ala and 1 Leu,
j) a variant of SEQ ID NO: 34 of between 15 to 23 amino acids, wherein the amino acid at position 1 is Met, the amino acid at position 2 is Val and wherein a contiguous stretch of 10 amino acids comprises at least 3 Ala and 2 Leu,
k) a variant of SEQ ID NO: 34 of between 15 to 23 amino acids, wherein the amino acid at position 1 is Met, the amino acid at position 2 is Val and wherein a contiguous stretch of 10 amino acids comprises at least 2 Ala and 3 Leu,
l) a variant of SEQ ID NO: 34 of between 15 to 23 amino acids, wherein the amino acid at position 1 is Met, the amino acid at position 2 is Val and wherein a contiguous stretch of 10 amino acids comprises at least 1 Ala and 4 Leu,
m) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Leu,
n) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu,
o) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu, and wherein the amino acids at the three last positions of the variant are Ala, Leu, Ala,
p) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Ala,
q) a variant of SEQ ID NO: 34 of between 15 to 23 amino acids, wherein the amino acid at position 1 is Met, the amino acid at position 2 is Lys and wherein a contiguous stretch of 10 amino acids comprises at least 4 Ala and 1 Leu,
r) a variant of SEQ ID NO: 34 of between 15 to 23 amino acids, wherein the amino acid at position 1 is Met, the amino acid at position 2 is Lys and wherein a contiguous stretch of 10 amino acids comprises at least 3 Ala and 2 Leu,
s) a variant of SEQ ID NO: 34 of between 15 to 23 amino acids, wherein the amino acid at position 1 is Met, the amino acid at position 2 is Lys and wherein a contiguous stretch of 10 amino acids comprises at least 2 Ala and 3 Leu,
t) a variant of SEQ ID NO: 34 of between 15 to 23 amino acids, wherein the amino acid at position 1 is Met, the amino acid at position 2 is Lys and wherein a contiguous stretch of 10 amino acids comprises at least 1 Ala and 4 Leu,
u) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Leu

In variants (a) to (d) of SEQ ID NO: 25, (a) and (b) of SEQ ID NO: 39, (a) and (b) of SEQ ID NO: 44 and of (a) to (u) of SEQ ID NO: 34 here above, the contiguous stretch is preferably 10 amino acids, more preferably 9 amino acids, even more preferably 8 amino acids and most preferably 7 amino acids.

In variants (a) to (d) of SEQ ID NO: 25, (a) and (b) of SEQ ID NO: 39, (a) and (b) of SEQ ID NO: 44 and of (a) to (u) of SEQ ID NO: 34 here above the contiguous stretch of amino acids comprises preferably at least 5 amino acids selected from Ala or Leu, more preferably at least 6 amino acids selected from Ala or Leu and most preferably at least 7 amino acids selected from Ala or Leu.

Variants of SEQ ID NO: 25, SEQ ID NO: 39, SEQ ID NO: 44, or SEQ ID NO: 34 of between 15 to 23 amino acids may comprise 15 amino acids, 17 amino acids, 18 amino acids, 19 amino acids, 20 amino acids, 21 amino acids, 22 amino acids or 23 amino acids.

The signal peptides encoded by the first polynucleotide described here above are herein referred to as the signal peptide according to the invention.

A “peptide” or “oligopeptide” is herein referred to as a molecule comprised of at least two amino acids arranged in a linear chain and joined together by peptide bonds between the carboxyl and amino groups of adjacent amino acid residues. The terms “peptide” and “oligopeptide” are considered synonymous (as is commonly recognized) and each term can be used interchangeably as the context requires. A “polypeptide” is herein referred to as a molecule comprising at least 40 amino acids.

In the context of the present invention, the term “signal peptide” is defined herein as a peptide that leads a polypeptide into the secretory pathway of the host cell. A signal sequence is usually, but not necessarily, present at the amino terminus of the polypeptide, fused in frame to the polypeptide. Between the signal peptide and the amino terminus of the polypeptide, a propeptide may be present. The signal sequence is usually, but not necessarily cleaved of the polypeptide during the secretion process to yield the mature polypeptide. The person skilled in the art knows how to identify a signal sequence. Various tools and ample literature are available. Examples that are not to be construed as limitations of the invention are:
A new method for predicting signal sequence cleavage sites. von Heijne G. Nucleic Acids Res. 1986 Jun. 11; 14(11):4683-90.
Identification of prokaryotic and eukaryotic signal peptides and prediction of their cleavage sites. Henrik Nielsen, Jacob Engelbrecht, Søren Brunak and Gunnar von Heijne. Protein Engineering, 10:1-6, 1997.
Locating proteins in the cell using TargetP, SignalP, and related tools. Olof Emanuelsson, Søren Brunak, Gunnar von Heijne, Henrik Nielsen. Nature Protocols 2, 953-971 (2007). website: http://www.cbs.dtu.dk/services/SignalP/

The term “propeptide” is defined herein as a peptide fused in frame to the amino terminus of a polypeptide. The resulting polypeptide is known as a propolypeptide and can be converted into a mature polypeptide by catalytic of autocatalytic cleavage of the propeptide from the propolypeptide.

A signal peptide and propeptide together are herein referred to as a “prepropeptide”, the signal sequence being fused in frame to the propeptide and the propeptide being fused in frame to the amino terminus of the polypeptide.

Signal peptides, propeptides and prepropeptides are in the art sometimes referred to as “leader sequences”.

The term “mature polypeptide” is defined herein as a polypeptide in its final form after translation, post-translational modifications such as N-terminal processing, C-terminal processing, glycosylation, phosphorylation and optional removal of leader sequences by cleavage.

In the context of the present invention the terms “polypeptide” and “protein” are identical and throughout the description of the present invention can be read interchangeably.

In the context of the present invention, the term “recombinant” refers to any genetic modification not exclusively involving naturally occurring processes and/or genetic modifications induced by subjecting the host cell to random mutagenesis. Consequently, combinations of recombinant and naturally occurring processes and/or genetic modifications induced by subjecting the host cell to random mutagenesis are construed as being recombinant. Preferably, recombinant genetic modification does not involve naturally occurring processes and/or genetic modifications induced by subjecting the host cell to random mutagenesis.

The term “operably linked” is defined herein as a configuration in which a control sequence is placed at an appropriate position relative to a coding sequence such that the control sequence directs the expression of the coding sequence.

The term “coding sequence” as defined herein is a sequence, which is transcribed into mRNA and translated into a polypeptide according to the invention. The boundaries of the coding sequence are generally determined by the ATG or other start codon at the 5′-side of the mRNA and a translation stop codon sequence terminating the open reading frame at the 3′-side of the mRNA. A coding sequence can include, but is not limited to, DNA, cDNA, and recombinant nucleic acid sequences.

The term “variant peptide” or “variant polypeptide” is defined herein as a peptide or polypeptide, respectively, comprising one or more alterations, such as substitutions, insertions, deletions and/or truncations of one or more specific amino acid residues at one or more specific positions in the peptide or polypeptide, respectively. Accordingly, a variant signal peptide is a signal peptide comprising one or more alterations, such as substitutions, insertions, deletions and/or truncations of one or more specific amino acid residues at one or more specific positions in the signal peptide.

The corresponding positions of the variant signal peptide according to the present invention are determined by alignment to a reference sequence such as signal peptides SEQ ID NO: 25, SEQ ID NO: 39, SEQ ID NO: 44, or SEQ ID NO: 34. Alignments or multi-alignments of peptides, polypeptides or polynucleotides, as applicable, can be made using methods known in the art. Such methods include, but are not limited to, ClustalW (Thompson et al, 1994, Nucleic Acid Research 22, 4673-4680), BLAST, GAP, MAP, MultiBLAST, and Smith Waterman.

The term “polynucleotide” is identical to the term “nucleic acid molecule” and can herein be read interchangeably. The term refers to a polynucleotide molecule, which is a ribonucleic acid (RNA) or deoxyribonucleic acid (DNA) molecule, either single stranded or double stranded. A polynucleotide may either be present in isolated form, or be comprised in recombinant nucleic acid molecules or vectors, or be comprised in a host cell.

The term “variant polynucleotide” is defined herein as a polynucleotide comprising one or more alterations, such as substitutions, insertions, deletions and/or truncations of one or more nucleotides at one or more specific positions in the polynucleotide.

The signal peptide according to the invention can be natively associated with the polypeptide of interest encoded by the second polynucleotide or can be foreign to the polypeptide of interest encoded by the second polynucleotide. Preferably, the signal peptide according to the invention is foreign to the polypeptide of interest encoded by the second polynucleotide. A variant signal peptide is herein defined as foreign to the polypeptide of interest encoded by the second polynucleotide.

A signal peptide natively associated with a polypeptide of interest may be replaced by a signal peptide according to the invention by physical replacement of the polynucleotide encoding the natively associated signal peptide with a signal peptide according to the invention by using standard molecular cloning techniques known in the art. Such methods are extensively described in Sambrook & Russell, Molecular Cloning: A Laboratory Manual, 3rd Ed., CSHL Press, Cold Spring Harbor, N.Y., 2001; and Ausubel et al., Current Protocols in Molecular Biology, Wiley InterScience, NY, 1995.

Alternatively, the signal peptide natively associated with the polypeptide of interest may be converted into a signal peptide according to the invention by site-specific mutagenesis of the polynucleotide encoding the natively associated signal peptide using methods known in the (see e.g. Sambrook & Russel, supra).

The signal peptide according to the invention can be native or foreign to the filamentous fungal host cell. Preferably, the signal peptide according to the invention is native to the filamentous fungal host cell.

Preferably, the process according to the invention produces at least 10% more, more preferable at least 25% more, even more preferably at least 50% more, even more preferably at least 75% more, even more preferably at least 100% more, even more preferably at least 200% more, most preferably at least 500% more of the recombinant polypeptide of interest encoded by the second polynucleotide linked in transitional reading frame with the first polynucleotide encoding a signal peptide according to the invention as compared to the polypeptide of interest encoded by the second polynucleotide linked in transitional reading frame with the polynucleotide encoding its native signal peptide, when cultivated under identical conditions.

The second polynucleotide, encoding a polypeptide of interest, may be provided for by general methods known to the person skilled in the art. Such methods are extensively described in Sambrook & Russell supra. Examples of said methods are following. When the sequence of the second polynucleotide is already known, or when the sequence of the polypeptide of interest encoded is already known, the polynucleotide may be isolated from a host cell that natively expresses the polynucleotide. Alternatively, the polynucleotide may be synthesized chemically. Codon optimization methods as e.g. described here below may be used for adaptation of the codon use a host cell of choice. If the sequence of the polypeptide is not known, the sequence may first be determined using methods known in the art (Sambrook & Russel, supra).

The polynucleotides herein combined or alone (i.e. the first polynucleotide linked in translational reading frame to a second polynucleotide, said second polynucleotide encoding a polypeptide of interest, said first polynucleotide encoding a signal peptide; or the first polynucleotide alone or the second polynucleotide alone) may be synthetic polynucleotides. The synthetic polynucleotides may be optimized in codon use, preferably according to the methods described in WO2006/077258 and/or PCT/EP2007/055943, which are herein incorporated by reference. PCT/EP2007/055943 addresses codon-pair optimization. Codon-pair optimisation is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence.

The polynucleotides herein combined or alone (i.e. the first polynucleotide linked in translational reading frame to a second polynucleotide, said second polynucleotide encoding a polypeptide of interest, said first polynucleotide encoding a signal peptide; or the first polynucleotide alone or the second polynucleotide alone) may comprise one or more introns.

Methods to link polynucleotides to each other in translational reading frame are known in the art as general cloning techniques (Sambrook & Russell, supra). Examples are digestion, ligation, PCR, chemical synthesis etc. Thus, the first polynucleotide can be linked in translational reading frame to a second polynucleotide by such methods known in the art.

The filamentous fungal host cell comprising a first polynucleotide linked in translational reading frame to a second polynucleotide, said second polynucleotide encoding a polypeptide of interest, said first polynucleotide encoding a signal peptide according to the invention can be constructed using methods known in the art. Preferably, said filamentous fungal host cell is constructed by a process comprising:
providing a suitable filamentous fungal host cell, and
transforming said host cell with said first polynucleotide linked in translational reading frame to said second polynucleotide.

Transformation of the host cell by introduction of a polynucleotide an expression vector or a nucleic acid construct into the cell is preferably performed by techniques well known in the art (see Sambrook & Russell; Ausubel, supra). Transformation may involve a process consisting of protoplast formation, transformation of the protoplasts, and regeneration of the cell wall in a manner known per se. Suitable procedures for transformation of Aspergillus cells are described in EP 238 023 and Yelton et al., 1984, Proceedings of the National Academy of Sciences USA 81:1470-1474. Suitable procedures for transformation of Aspergillus and other filamentous fungal host cells using Agrobacterium tumefaciens are described in e.g. De Groot et al., Agrobacterium tumefaciens-mediated transformation of filamentous fungi. Nat Biotechnol. 1998, 16:839-842. Erratum in: Nat Biotechnol 1998 16:1074. A suitable method of transforming Fusarium species is described by Malardier et al., 1989, Gene 78:147156 or in WO 96/00787. Other methods can be applied such as a method using biolistic transformation as described in: Christiansen et al., Biolistic transformation of the obligate plant pathogenic fungus, Erysiphe graminis f. sp. hordei. 1995, Curr Genet. 29:100-102. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

The filamentous fungal host cells according to the present invention are cultivated in a nutrient medium suitable for production of the recombinant polypeptide of interest using methods known in the art. For example, the cells may be cultivated by shake flask cultivation, small-scale or large-scale fermentation (including continuous, batch, fedbatch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art (see, e.g., Bennett, J. W. and LaSure, L., eds., More Gene Manipulations in Fungi, Academic Press, CA, 1991). Suitable media are available from commercial suppliers or may be prepared using published compositions (e.g., in catalogues of the American Type Culture Collection). If the polypeptide is secreted into the nutrient medium, the polypeptide can be recovered directly from the culture medium. If the polypeptide is not secreted, it is recovered from cell lysates.

The recombinant polypeptide of interest produced may be recovered from the culture medium by the methods known in the art. For example, the polypeptide may be recovered from the culture medium by conventional procedures including, but not limited to, centrifugation, filtration, extraction, spray-drying, evaporation, or precipitation.

The recombinant polypeptide of interest may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

The recombinant polypeptide of interest may be detected using methods known in the art that are specific for the polypeptides. These detection methods may include use of specific antibodies, high performance liquid chromatography, capillary chromatography, electrophoresis, formation of an enzyme product, or disappearance of an enzyme substrate.

The host cell according to the invention is a filamentous fungal host cell. “Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Sporotrichum, Schizophyllum, Talaromyces, Thermoascus, Thielavia, Tolypocladium, and Trichoderma.

Preferred filamentous fungal cells belong to a species of an Acremonium, Aspergillus, Chrysosporium, Myceliophthora, Penicillium, Sporotrichum, Talaromyces, Thielavia or Trichoderma genus, and most preferably a species of Acremonium alabamensis, Aspergillus niger, Aspergillus awamori, Aspergillus foetidus, Aspergillus sojae, Aspergillus fumigatus, Aspergillus oryzae, Chrysosporium lucknowense, Myceliophthora thermophila, Sporotrichum cellulophilum, Thielavia terrestris, Trichoderma reesei, Talaromyces emersonii or Penicillium chrysogenum.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL) Aspergillus niger CBS513.88, Aspergillus oryzae ATCC 20423, IFO 4177, ATCC 1011, ATCC 9576, ATCC14488-14491, ATCC 11601, ATCC12892, P. chrysogenum CBS 455.95, Penicillium citrinum ATCC 38065, Penicillium chrysogenum P2, Acremonium chrysogenum ATCC 36225 or ATCC 48272, Trichoderma reesei ATCC 26921 or ATCC 56765 or ATCC 26921, Aspergillus sojae ATCC11906, Chrysosporium lucknowense ATCC44006, Talaromyces emersonii CBS393.64 or CBS814.70 and derivatives thereof.

Optionally, the host cell comprises an elevated unfolded protein response (UPR) compared to the wild type cell to enhance production abilities of a polypeptide of interest. UPR may be increased by techniques described in US2004/0186070A1 and/or US2001/0034045A1 and/or WO01/72783A2 and/or WO2005/123763. More specifically, the protein level of HAC1 and/or IRE1 and/or PTC2 has been modulated, and/or the SEC61 protein has been engineered in order to obtain a host cell having an elevated UPR.

Alternatively, or in combination with an elevated UPR, the host cell is genetically modified to obtain a phenotype displaying lower protease expression and/or protease secretion compared to the wild-type cell in order to enhance production abilities of a polypeptide of interest. Such phenotype may be obtained by deletion and/or modification and/or inactivation of a transcriptional regulator of expression of proteases. Such a transcriptional regulator is e.g. prtT. Lowering expression of proteases by modulation of prtT may be performed by techniques described in US2004/0191864A1.

Alternatively, or in combination with an elevated UPR and/or a phenotype displaying lower protease expression and/or protease secretion, the host cell displays an oxalate deficient phenotype in order to enhance the yield of production of a polypeptide of interest. An oxalate deficient phenotype may be obtained by techniques described in WO2004/070022A2.

Alternatively, or in combination with an elevated UPR and/or a phenotype displaying lower protease expression and/or protease secretion and/or oxalate deficiency, the host cell displays a combination of phenotypic differences compared to the wild cell to enhance the yield of production of the polypeptide of interest. These differences may include, but are not limited to, lowered expression of glucoamylase and/or neutral alpha-amylase A and/or neutral alpha-amylase B, protease, and oxalic acid hydrolase. Said phenotypic differences displayed by the host cell may be obtained by genetic modification according to the techniques described in US2004/0191864A1.

Alternatively, or in combination with an elevated UPR and/or a phenotype displaying lower protease expression and/or protease secretion and/or oxalate deficiency and a combination of phenotypic differences compared to the wild cell to enhance the yield of production of the polypeptide of interest, the host cell displays a deficiency in toxin genes, disabling the ability of the filamentous fungal host cell to express toxins. Such toxins include, but are not limited to, ochratoxins, fumonisins, cyclapiazonic acid, 3-nitropropionic acid, emodin, malformin, aflatoxins and secalonic acids. Such deficiency is preferably such as described in WO2000/039322.

The polypeptide of interest may be any polypeptide having a biological activity of interest. The polypeptide may native or may be heterologous to the host cell. A heterologous polypeptide is defined herein as a polypeptide which is not native to the host cell, or a native polypeptide in which structural modifications were made to alter the polypeptide. The polypeptide may be a collagen or gelatin, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein natively involved in secretion process, protein involved in folding process, chaperone, peptide amino acid transporter, glycosylation factor, transcription factor, oligopeptide, natively intracellular protein. The natively intracellular protein may be an enzyme such as, a protease, ceramidases, epoxide hydrolase, aminopeptidase, acylases, aldolase, hydroxylase, aminopeptidase, lipase. The recombinant polypeptide of interest is preferably an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an asparaginase, aminopeptidase, amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, proteolytic enzyme, invertase, laccase, lipase, mannosidase, mutanase, oxidase, pectinolytic enzyme, peroxidase, phospholipase, polyphenoloxidase, ribonuclease, transglutaminase, or glucose oxidase, hexose oxidase, monooxygenase.

Polypeptides further include naturally occurring allelic and engineered variations of the above-mentioned polypeptides.

According to the present invention, the polypeptide of interest can also be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

The hybrid polypeptides may comprise a combination of partial or complete polypeptide sequences obtained from at least two different polypeptides wherein one or more may be heterologous to the host cell.

The process according to the present invention is conveniently used to produce a recombinant polypeptide of interest.

Accordingly, in a second aspect the present invention relates to the recombinant polypeptide of interest produced by the process according to the first aspect of the invention. Preferably, said polypeptide is an enzyme as described here above.

The present invention further relates to an intermediate product, namely the polypeptide of interest encoded by a first polynucleotide linked in translational reading frame to a second polynucleotide, said second polynucleotide encoding a polypeptide of interest, said first polynucleotide encoding a signal peptide according to the invention. The polypeptide of interest is preferably the polypeptide of interest described in the first aspect of the invention.

Preferably, the signal peptide is one selected from the group consisting of:
a) SEQ ID NO: 25,
b) a variant of SEQ ID NO: 25 of between 15 and 23 amino acids, wherein at least 8 amino acids are identical to the first 10 amino acids of SEQ ID NO: 25 at corresponding positions,
c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
d) SEQ ID NO: 39,
e) a variant of SEQ ID NO: 39 of between 15 and 23 amino acids, wherein at least 8 amino acids are identical to the first 10 amino acids of SEQ ID NO: 39 at corresponding positions,
f) a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
g) SEQ ID NO: 44,
h) a variant of SEQ ID NO: 44 of between 15 and 23 amino acids, wherein at least 8 amino acids are identical to the first 10 amino acids of SEQ ID NO: 44 at corresponding positions,
i) a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
j) SEQ ID NO: 34,
k) a variant of SEQ ID NO: 34 of between 15 and 23 amino acids, wherein at least 8 amino acids are identical to the first 10 amino acids of SEQ ID NO: 34 at corresponding positions,
l) a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to an embodiment, in the intermediate product, the signal peptide is SEQ ID NO: 25.

According to another embodiment, in the intermediate product, the signal peptide is a variant of SEQ ID NO: 25 of between 15 and 23 amino acids, wherein at least 8 amino acids are identical to the first 10 amino acids of SEQ ID NO: 25 at corresponding positions.

According to another embodiment, in the intermediate product, the signal peptide is a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, in the intermediate product, the signal peptide is SEQ ID NO: 39.

According to another embodiment, in the intermediate product, the signal peptide is a variant of SEQ ID NO: 39 of between 15 and 23 amino acids, wherein at least 8 amino acids are identical to the first 10 amino acids of SEQ ID NO: 39 at corresponding positions.

According to another embodiment, in the intermediate product, the signal peptide is a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, in the intermediate product, the signal peptide is SEQ ID NO: 44.

According to another embodiment, in the intermediate product, the signal peptide is a variant of SEQ ID NO: 44 of between 15 and 23 amino acids, wherein at least 8 amino acids are identical to the first 10 amino acids of SEQ ID NO: 44 at corresponding positions.

According to another embodiment, in the intermediate product, the signal peptide is a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, in the intermediate product, the signal peptide is SEQ ID NO: 34.

According to another embodiment, in the intermediate product, the signal peptide is a variant of SEQ ID NO: 34 of between 15 and 23 amino acids, wherein at least 8 amino acids are identical to the first 10 amino acids of SEQ ID NO: 34 at corresponding positions. According to another embodiment, in the intermediate product, the signal peptide is a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

Preferably, in the intermediate product, when the signal peptide is
(a): SEQ ID NO: 25,
(b): a variant of SEQ ID NO: 25 of between 15 and 23 amino acids, wherein at least 8 amino acids are identical to the first 10 amino acids of SEQ ID NO: 25 at corresponding positions, or
(c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   the polypeptide of interest is not a pectin methyl esterase, more preferably the polypeptide of interest is not a pectin methyl esterase from Erwinia chrysanthemi. More preferably, when the signal peptide is (b) or (c), the polypeptide of interest is not a pectin methyl esterase, even more preferably the polypeptide of interest is not a pectin methyl esterase from Erwinia chrysanthemi.

Preferably, the first polynucleotide when encoding SEQ ID NO: 25, is a polynucleotide according to SEQ ID NO: 29. Preferably, the first polynucleotide when encoding SEQ ID NO: 39, is a polynucleotide according to SEQ ID NO: 38. Preferably, the first polynucleotide when encoding SEQ ID NO: 44, is a polynucleotide according to SEQ ID NO: 43. Preferably, the first polynucleotide when encoding SEQ ID NO: 34, is a polynucleotide according to SEQ ID NO: 33.

Preferably, the variant of SEQ ID NO: 25 of between 15 and 23 amino acids, wherein at least 8 amino acids are identical to the first 10 amino acids of SEQ ID NO: 25 at corresponding positions, is a variant wherein at least 9 amino acids are identical to the first 10 amino acids of SEQ ID NO: 25 at corresponding positions. More preferably, 10 amino acids are identical to the first 10 amino acids of SEQ ID NO: 25 at corresponding positions.

Preferably, the variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu,
c) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu, and wherein the amino acids of the last three positions of the variant are Ala, Leu and Ala.

Preferably, the variant of SEQ ID NO: 39 of between 15 and 23 amino acids, wherein at least 8 amino acids are identical to the first 10 amino acids of SEQ ID NO: 39 at corresponding positions, is a variant wherein at least 9 amino acids are identical to the first 10 amino acids of SEQ ID NO: 39 at corresponding positions. More preferably, 10 amino acids are identical to the first 10 amino acids of SEQ ID NO: 39 at corresponding positions.

Preferably, the variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 39 of between 15 to 23 amino acids, wherein the amino acid at position 1 is Met, the amino acid at position 2, 3 and/or 5 is Val or Lys and wherein a contiguous stretch of 10 amino acids comprises at least 5 amino acids selected from Ala or Leu,
b) a variant of SEQ ID NO: 39 of between 15 to 23 amino acids, wherein the amino acid at position 1 is Met, the amino acid at position 2 is Val and the amino acid at position 5 is Lys and wherein a contiguous stretch of 10 amino acids comprises at least 5 amino acids selected from Ala or Leu.

Preferably, the variant of SEQ ID NO: 44 of between 15 and 23 amino acids, wherein at least 8 amino acids are identical to the first 10 amino acids of SEQ ID NO: 44 at corresponding positions, is a variant wherein at least 9 amino acids are identical to the first 10 amino acids of SEQ ID NO: 44 at corresponding positions. More preferably, 10 amino acids are identical to the first 10 amino acids of SEQ ID NO: 44 at corresponding positions.

Preferably, the variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 44 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 44 of between 15 to 23 amino acids, wherein the amino acid at position 1 is Met, the amino acid at position 2 is Val and the amino acid at position 4 is Lys and wherein a contiguous stretch of 10 amino acids comprises at least 5 amino acids selected from Ala or Leu.

Preferably, the variant of SEQ ID NO: 34 of between 15 and 23 amino acids, wherein at least 8 amino acids are identical to the first 10 amino acids of SEQ ID NO: 34 at corresponding positions, is a variant wherein at least 9 amino acids are identical to the first 10 amino acids of SEQ ID NO: 34 at corresponding positions. More preferably, 10 amino acids are identical to the first 10 amino acids of SEQ ID NO: 34 at corresponding positions.

Preferably, the variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu,
b) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu,
c) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu, and wherein the amino acids of the last three positions of the variant are Ala, Leu and Ala,
e) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu,
f) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids of the last three positions of the variant are Ala, Leu and Ala,
g) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids at the three last positions of the variant are Ala, Leu, Ala,
h) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Ala,
i) a variant of SEQ ID NO: 34 of between 15 to 23 amino acids, wherein the amino acid at position 1 is Met, the amino acid at position 2 is Val and wherein a contiguous stretch of 10 amino acids comprises at least 4 Ala and 1 Leu,
j) a variant of SEQ ID NO: 34 of between 15 to 23 amino acids, wherein the amino acid at position 1 is Met, the amino acid at position 2 is Val and wherein a contiguous stretch of 10 amino acids comprises at least 3 Ala and 2 Leu,
k) a variant of SEQ ID NO: 34 of between 15 to 23 amino acids, wherein the amino acid at position 1 is Met, the amino acid at position 2 is Val and wherein a contiguous stretch of 10 amino acids comprises at least 2 Ala and 3 Leu,
l) a variant of SEQ ID NO: 34 of between 15 to 23 amino acids, wherein the amino acid at position 1 is Met, the amino acid at position 2 is Val and wherein a contiguous stretch of 10 amino acids comprises at least 1 Ala and 4 Leu,
m) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Leu,
n) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu,
o) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu, and wherein the amino acids at the three last positions of the variant are Ala, Leu, Ala,
p) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Ala,
q) a variant of SEQ ID NO: 34 of between 15 to 23 amino acids, wherein the amino acid at position 1 is Met, the amino acid at position 2 is Lys and wherein a contiguous stretch of 10 amino acids comprises at least 4 Ala and 1 Leu,
r) a variant of SEQ ID NO: 34 of between 15 to 23 amino acids, wherein the amino acid at position 1 is Met, the amino acid at position 2 is Lys and wherein a contiguous stretch of 10 amino acids comprises at least 3 Ala and 2 Leu,
s) a variant of SEQ ID NO: 34 of between 15 to 23 amino acids, wherein the amino acid at position 1 is Met, the amino acid at position 2 is Lys and wherein a contiguous stretch of 10 amino acids comprises at least 2 Ala and 3 Leu,
t) a variant of SEQ ID NO: 34 of between 15 to 23 amino acids, wherein the amino acid at position 1 is Met, the amino acid at position 2 is Lys and wherein a contiguous stretch of 10 amino acids comprises at least 1 Ala and 4 Leu,
u) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Leu.

In variants (a) to (d) of SEQ ID NO: 25, (a) and (b) of SEQ ID NO: 39, (a) and (b) of SEQ ID NO: 44 and of (a) to (u) of SEQ ID NO: 34 here above, the contiguous stretch is preferably 10 amino acids, more preferably 9 amino acids, even more preferably 8 amino acids and most preferably 7 amino acids.

In variants (a) to (d) of SEQ ID NO: 25, (a) and (b) of SEQ ID NO: 39, (a) and (b) of SEQ ID NO: 44 and of (a) to (u) of SEQ ID NO: 34 here above the contiguous stretch of amino acids comprises preferably at least 5 amino acids selected from Ala or Leu, more preferably at least 6 amino acids selected from Ala or Leu and most preferably at least 7 amino acids selected from Ala or Leu.

Variants of SEQ ID NO: 25, SEQ ID NO: 39, SEQ ID NO: 44, or SEQ ID NO: 34 of between 15 to 23 amino acids may comprise 15 amino acids, 16 amino acids, 17 amino acids, 18 amino acids, 19 amino acids, 20 amino acids, 21 amino acids, 22 amino acids or 23 amino acids.

In a third aspect, the present invention relates to a recombinant expression construct comprising: a first polynucleotide linked in translational reading frame to a second polynucleotide, said second polynucleotide encoding a polypeptide of interest, said first polynucleotide encoding a signal peptide according to the invention. The polypeptide of interest is preferably the polypeptide of interest described in the first aspect of the invention. Preferably, when the signal peptide is
(a): SEQ ID NO: 25,
(b): a variant of SEQ ID NO: 25 of between 15 and 23 amino acids, wherein at least 8 amino acids are identical to the first 10 amino acids of SEQ ID NO: 25 at corresponding positions, or
(c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   the polypeptide of interest is not a pectin methyl esterase, more preferably the polypeptide of interest is not a pectin methyl esterase from Erwinia chrysanthemi. More preferably, when the signal peptide is (b) or (c), the polypeptide of interest is not a pectin methyl esterase, even more preferably the polypeptide of interest is not a pectin methyl esterase from Erwinia chrysanthemi.

Preferably, the first polynucleotide when encoding SEQ ID NO: 25, is a polynucleotide according to SEQ ID NO: 29. Preferably, the first polynucleotide when encoding SEQ ID NO: 39, is a polynucleotide according to SEQ ID NO: 38. Preferably, the first polynucleotide when encoding SEQ ID NO: 44, is a polynucleotide according to SEQ ID NO: 43. Preferably, the first polynucleotide when encoding SEQ ID NO: 34, is a polynucleotide according to SEQ ID NO: 33.

The present invention further relates to said recombinant expression construct further comprising a promoter operably linked to a first polynucleotide linked in translational reading frame to a second polynucleotide, said second polynucleotide encoding a polypeptide of interest, said first polynucleotide encoding a signal peptide according to the invention. The polypeptide of interest is preferably the polypeptide of interest described in the first aspect of the invention.

The present invention further relates to a recombinant expression vector comprising the expression constructs described here above.

The term “nucleic acid construct” is herein referred to as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence, wherein said control sequences are operably linked to said coding sequence.

The term “control sequences” is defined herein to include all components, which are necessary or advantageous for the expression of mRNA and/or a polypeptide, either in vitro or in a host cell. Each control sequence may be native or foreign to the nucleic acid sequence encoding the polypeptide. Such control sequences include, but are not limited to, a Shine-Delgarno sequence, optimal translation initiation sequences (as described in Kozak, 1991, J. Biol. Chem. 266:19867-19870), a polyadenylation sequence, a promoter, and a transcription terminator. At a minimum, the control sequences include a promoter, and transcriptional and translational stop signals. Control sequences may be optimized to their specific purpose. Preferably, the DNA construct comprises a promoter DNA sequence, a coding sequence in operative association with said promoter DNA sequence and control sequences such as:
one translational termination sequence orientated in 5′ towards 3′ direction selected from the following list of sequences: TAAG, TAGA and TAAA, preferably TAAA, and/or
one translational initiator coding sequence orientated in 5′ towards 3′ direction selected from the following list of sequences: GCTACCCCC; GCTACCTCC; GCTACCCTC; GCTACCTTC; GCTCCCCCC; GCTCCCTCC; GCTCCCCTC; GCTCCCTTC; GCTGCCCCC; GCTGCCTCC; GCTGCCCTC; GCTGCCTTC; GCTTCCCCC; GCTTCCTCC; GCTTCCCTC; and GCTTCCTTC, preferably GCT TCC TTC, and/or
one translational initiator sequence selected from the following list of sequences: 5′-mwChkyCAAA-3′; 5′-mwChkyCACA-3′ or 5′-mwChkyCAAG-3′, using ambiguity codes for nucleotides: m (NC); w (NT); y (C/T); k (G/T); h (A/C/T), preferably 5′-CACCGTCAAA-3′ or 5′-CGCAGTCAAG-3′.

In the context of this invention, the term “translational initiator coding sequence” is defined as the nine nucleotides immediately downstream of the initiator or start codon of the open reading frame of a DNA coding sequence. The initiator or start codon encodes for the AA methionine. The initiator codon is typically ATG, but may also be any functional start codon such as GTG.

In the context of this invention, the term “translational termination sequence” is defined as the four nucleotides starting from the translational stop codon at the 3′ end of the open reading frame or nucleotide coding sequence and oriented in 5′ towards 3′ direction.

In the context of this invention, the term “translational initiator sequence” is defined as the ten nucleotides immediately upstream of the initiator or start codon of the open reading frame of a DNA sequence coding for a polypeptide. The initiator or start codon encodes for the AA methionine. The initiator codon is typically ATG, but may also be any functional start codon such as GTG. It is well known in the art that uracil, U, replaces the deoxynucleotide thymine, T, in RNA.

The control sequences may be provided with linkers for the purpose of introducing specific restriction sites facilitating ligation of the control sequences with the coding region of the nucleic acid sequence encoding a polypeptide. The control sequence may be an appropriate promoter sequence, a nucleic acid sequence, which is recognized by a host cell for expression of the nucleic acid sequence. The promoter sequence contains transcriptional control sequences, which mediate the expression of the polypeptide. The promoter may be any nucleic acid sequence, which shows transcriptional activity in the cell including mutant, truncated, and hybrid promoters, and may be obtained from genes encoding extracellular or intracellular polypeptides either homologous or heterologous to the cell.

The control sequence may also be a suitable transcription terminator sequence, a sequence recognized by a filamentous fungal cell to terminate transcription. The terminator sequence is operably linked to the 3′-terminus of the nucleic acid sequence encoding the polypeptide. Any terminator, which is functional in the cell, may be used in the present invention.

Preferred terminators for filamentous fungal cells are obtained from the genes encoding A. oryzae TAKA amylase, A. niger glucoamylase (glaA), A. nidulans anthranilate synthase, A. niger alpha-glucosidase, trpC gene and Fusarium oxysporum trypsin-like protease.

The control sequence may also be a polyadenylation sequence, a sequence which is operably linked to the 3′-terminus of the nucleic acid sequence and which, when transcribed, is recognized by the filamentous fungal cell as a signal to add polyadenosine residues to transcribed mRNA. Any polyadenylation sequence, which is functional in the cell, may be used in the present invention.

Preferred polyadenylation sequences for filamentous fungal cells are obtained from the genes encoding A. oryzae TAKA amylase, A. niger glucoamylase, A. nidulans anthranilate synthase, Fusarium oxysporum trypsin-like protease and A. niger alpha-glucosidase.

The term “promoter” is defined herein as a DNA sequence that binds RNA polymerase and directs the polymerase to the correct downstream transcriptional start site of a nucleic acid sequence encoding a biological compound to initiate transcription. RNA polymerase effectively catalyzes the assembly of messenger RNA complementary to the appropriate DNA strand of a coding region. The term “promoter” will also be understood to include the 5′-non-coding region (between promoter and translation start) for translation after transcription into mRNA, cis-acting transcription control elements such as enhancers, and other nucleotide sequences capable of interacting with transcription factors. The promoter may be any appropriate promoter sequence suitable for a eukaryotic or prokaryotic host cell, which shows transcriptional activity, including mutant, truncated, and hybrid promoters, and may be obtained from genes encoding extra-cellular or intracellular polypeptides either homologous (native) or heterologous (foreign) to the cell. The promoter may be a constitutive or inducible promoter. Examples of inducible promoters that can be used are a starch-, copper-, oleic acid-inducible promoters. The promoter may be selected from the group, which includes but is not limited to promoters obtained from the genes encoding A. oryzae TAKA amylase, Rhizomucor miehei aspartic proteinase, A. niger neutral alpha-amylase, A. niger acid stable alpha-amylase, A. niger or A. awamori glucoamylase (glaA), R. miehei lipase, A. oryzae alkaline protease, A. oryzae triose phosphate isomerase, A. nidulans acetamidase, the NA2-tpi promoter (a hybrid of the promoters from the genes encoding A. niger neutral alpha-amylase and A. oryzae triose phosphate isomerase), and mutant, truncated, and hybrid promoters thereof. Particularly preferred promoters for use in filamentous fungal cells are a promoter, or a functional part thereof, from a protease gene; e.g., from the F. oxysporum trypsin-like protease gene (U.S. Pat. No. 4,288,627), A. oryzae alkaline protease gene (alp), A. niger pacA gene, A. oryzae alkaline protease gene, A. oryzae neutral metalloprotease gene, A. niger aspergillopepsin protease pepA gene, or F. venenatum trypsin gene, A. niger aspartic protease pepB gene. Other preferred promoters are the promoters described in WO2006/092396 and WO2005/100573, which are herein incorporated by reference.

When the recombinant polypeptide of interest is a chimeric polypeptide, being comprised of two or more (parts of) polypeptides, the person skilled in the art knows how to construct these and other chimeric polynucleotide constructs using methods known in the art.

In order to facilitate expression and/or translation, the polynucleotide or the nucleic acid construct according to the invention may be comprised in an expression vector such that the polynucleotide of the invention is operably linked to the appropriate control sequences for expression and/or translation in vitro, or in prokaryotic or eukaryotic host cells.

The recombinant expression vector may be any vector (e.g., a plasmid or virus), which can be conveniently subjected to recombinant DNA procedures and can bring about the expression of the nucleic acid sequence encoding the polypeptide. The choice of the vector will typically depend on the compatibility of the vector with the host cell into which the vector is to be introduced. The vectors may be linear or closed circular plasmids. The vector may be an autonomously replicating vector, i.e., a vector, which exists as an extra-chromosomal entity, the replication of which is independent of chromosomal replication, e.g., a plasmid, an extra-chromosomal element, a mini-chromosome, or an artificial chromosome. An autonomously maintained cloning vector may comprise the AMA1-sequence (see e.g. Aleksenko and Clutterbuck (1997), Fungal Genet. Biol. 21: 373-397).

Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell. In a preferred embodiment of the invention, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the cell. Linearization is preferably performed such that at least one but preferably either end of the cloning vector is flanked by sequences homologous to the target locus. The length of the homologous sequences flanking the target locus is preferably at least 30 bp, preferably at least 50 bp, preferably at least 0.1 kb, even preferably at least 0.2 kb, more preferably at least 0.5 kb, even more preferably at least 1 kb, most preferably at least 2 kb. Preferably, the efficiency of targeted integration into the genome of the host cell, i.e. integration in a predetermined target locus, is increased by augmented homologous recombination abilities of the host cell. Such phenotype of the cell preferably involves a deficient ku70 gene as described in WO2005/095624. WO2005/095624 discloses a preferred method to obtain a filamentous fungal cell comprising increased efficiency of targeted integration. Preferably, the homologous flanking DNA sequences in the cloning vector, which are homologous to the target locus, are derived from a highly expressed locus meaning that they are derived from a gene, which is capable of high expression level in the host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed fungal genes are given by way of example: the amylase, glucoamylase, alcohol dehydrogenase, xylanase, glyceraldehyde-phosphate dehydrogenase or cellobiohydrolase (cbh) genes from Aspergilli or Trichoderma. Most preferred highly expressed genes for these purposes are a glucoamylase gene, preferably an A. niger glucoamylase gene, an A. oryzae TAKA-amylase gene, an A. nidulans gpdA gene, a Trichoderma reesei cbh gene, preferably cbh1.

More than one copy of a nucleic acid sequence may be inserted into the cell to increase production of the gene product. This can be done, preferably by integrating into its genome copies of the DNA sequence, more preferably by targeting the integration of the DNA sequence at one of the highly expressed locus defined in the former paragraph. Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase even more the number of copies of the DNA sequence to be over expressed the technique of gene conversion as described in WO98/46772 may be used.

The vector system may be a single vector or plasmid or two or more vectors or plasmids, which together contain the total DNA to be introduced into the genome of the host cell, or a transposon.

The vectors preferably contain one or more selectable markers, which permit easy selection of transformed cells. A selectable marker is a gene the product of which provides for biocide or viral resistance, resistance to heavy metals, prototrophy to auxotrophs, and the like. A selectable marker for use in a filamentous fungal cell may be selected from the group including, but not limited to, amdS (acetamidase), argB (ornithine carbamoyltransferase), bar (phosphinothricinacetyltransferase), bleA (phleomycin binding), hygB (hygromycinphosphotransferase), niaD (nitrate reductase), pyrG (orotidine-5′-phosphate decarboxylase), sC (sulfate adenyltransferase), and trpC (anthranilate synthase), as well as equivalents from other species. Preferred for use in an Aspergillus and Penicillium cell are the amdS (EP 635574 B1, WO 97/06261) and pyrG genes of A. nidulans or A. oryzae and the bar gene of Streptomyces hygroscopicus. More preferably an amdS gene is used, even more preferably an amdS gene from A. nidulans or A. niger. A most preferred selection marker gene is the A. nidulans amdS coding sequence fused to the A. nidulans gpdA promoter (see EP 635574 B1). Other preferred AmdS markers are those described in WO2006/040358. AmdS genes from other filamentous fungi may also be used (WO 97/06261).

The procedures used to ligate the elements described above to construct the recombinant expression vectors of the present invention are well known to one skilled in the art (see, e.g. Sambrook & Russell, supra).

In a fourth aspect, the present invention relates to a recombinant filamentous fungal host cell comprising the expression construct according to the third aspect of the invention, or comprising the expression vector according to the third aspect of the invention. Said filamentous fungal host cell is preferably a cell as described earlier herein. Said filamentous fungal host cell can be constructed using methods known in the art. Preferably, said filamentous fungal host cell is constructed by a process comprising:
providing a suitable filamentous fungal host cell, and
transforming said host cell with the expression construct according to the third aspect of the invention, or with the expression vector according to the third aspect of the invention.

Transformation of the filamentous fungal host cell is preferably performed as described earlier herein.

In a fifth aspect, the present invention relates to the use of a signal peptide according to the invention for the production of a recombinant polypeptide of interest. Accordingly, the signal peptide is preferably selected from the group consisting of:
a) SEQ ID NO: 25,
b) a variant of SEQ ID NO: 25 of between 15 and 23 amino acids, wherein at least 8 amino acids are identical to the first 10 amino acids of SEQ ID NO: 25 at corresponding positions,
c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
d) SEQ ID NO: 39,
e) a variant of SEQ ID NO: 39 of between 15 and 23 amino acids, wherein at least 8 amino acids are identical to the first 10 amino acids of SEQ ID NO: 39 at corresponding positions,
f) a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
g) SEQ ID NO: 44,
h) a variant of SEQ ID NO: 44 of between 15 and 23 amino acids, wherein at least 8 amino acids are identical to the first 10 amino acids of SEQ ID NO: 44 at corresponding positions,
i) a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
j) SEQ ID NO: 34,
k) a variant of SEQ ID NO: 34 of between 15 and 23 amino acids, wherein at least 8 amino acids are identical to the first 10 amino acids of SEQ ID NO: 34 at corresponding positions,
l) a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to an embodiment, the signal peptide is SEQ ID NO: 25.

According to another embodiment, in the intermediate product, the signal peptide is a variant of SEQ ID NO: 25 of between 15 and 23 amino acids, wherein at least 8 amino acids are identical to the first 10 amino acids of SEQ ID NO: 25 at corresponding positions.

According to another embodiment, the signal peptide is a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, the signal peptide is SEQ ID NO: 39.

According to another embodiment, the signal peptide is a variant of SEQ ID NO: 39 of between 15 and 23 amino acids, wherein at least 8 amino acids are identical to the first 10 amino acids of SEQ ID NO: 39 at corresponding positions.

According to another embodiment, the signal peptide is a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, the signal peptide is SEQ ID NO: 44.

According to another embodiment, the signal peptide is a variant of SEQ ID NO: 44 of between 15 and 23 amino acids, wherein at least 8 amino acids are identical to the first 10 amino acids of SEQ ID NO: 44 at corresponding positions.

According to another embodiment, the signal peptide is a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, the signal peptide is SEQ ID NO: 34.

According to another embodiment, the signal peptide is a variant of SEQ ID NO: 34 of between 15 and 23 amino acids, wherein at least 8 amino acids are identical to the first 10 amino acids of SEQ ID NO: 34 at corresponding positions.

According to another embodiment, the signal peptide is a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

Preferably, when the signal peptide is
(a): SEQ ID NO: 25,
(b): a variant of SEQ ID NO: 25 of between 15 and 23 amino acids, wherein at least 8 amino acids are identical to the first 10 amino acids of SEQ ID NO: 25 at corresponding positions, or
(c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   the polypeptide of interest is not a pectin methyl esterase, more preferably the polypeptide of interest is not a pectin methyl esterase from Erwinia chrysanthemi. More preferably, when the signal peptide is (b) or (c), the polypeptide of interest is not a pectin methyl esterase, even more preferably the polypeptide of interest is not a pectin methyl esterase from Erwinia chrysanthemi.

Preferably, the variant of SEQ ID NO: 25 of between 15 and 23 amino acids, wherein at least 8 amino acids are identical to the first 10 amino acids of SEQ ID NO: 25 at corresponding positions, is a variant wherein at least 9 amino acids are identical to the first 10 amino acids of SEQ ID NO: 25 at corresponding positions. More preferably, 10 amino acids are identical to the first 10 amino acids of SEQ ID NO: 25 at corresponding positions.

Preferably, the variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu,
c) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu, and wherein the amino acids of the last three positions of the variant are Ala, Leu and Ala.

Preferably, the variant of SEQ ID NO: 39 of between 15 and 23 amino acids, wherein at least 8 amino acids are identical to the first 10 amino acids of SEQ ID NO: 39 at corresponding positions, is a variant wherein at least 9 amino acids are identical to the first 10 amino acids of SEQ ID NO: 39 at corresponding positions. More preferably, 10 amino acids are identical to the first 10 amino acids of SEQ ID NO: 39 at corresponding positions.

Preferably, the variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 39 of between 15 to 23 amino acids, wherein the amino acid at position 1 is Met, the amino acid at position 2, 3 and/or 5 is Val or Lys and wherein a contiguous stretch of 10 amino acids comprises at least 5 amino acids selected from Ala or Leu,
b) a variant of SEQ ID NO: 39 of between 15 to 23 amino acids, wherein the amino acid at position 1 is Met, the amino acid at position 2 is Val and the amino acid at position 5 is Lys and wherein a contiguous stretch of 10 amino acids comprises at least 5 amino acids selected from Ala or Leu.

Preferably, the variant of SEQ ID NO: 44 of between 15 and 23 amino acids, wherein at least 8 amino acids are identical to the first 10 amino acids of SEQ ID NO: 44 at corresponding positions, is a variant wherein at least 9 amino acids are identical to the first 10 amino acids of SEQ ID NO: 44 at corresponding positions. More preferably, 10 amino acids are identical to the first 10 amino acids of SEQ ID NO: 44 at corresponding positions.

Preferably, the variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 44 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 44 of between 15 to 23 amino acids, wherein the amino acid at position 1 is Met, the amino acid at position 2 is Val and the amino acid at position 4 is Lys and wherein a contiguous stretch of 10 amino acids comprises at least 5 amino acids selected from Ala or Leu.

Preferably, the variant of SEQ ID NO: 34 of between 15 and 23 amino acids, wherein at least 8 amino acids are identical to the first 10 amino acids of SEQ ID NO: 34 at corresponding positions, is a variant wherein at least 9 amino acids are identical to the first 10 amino acids of SEQ ID NO: 34 at corresponding positions. More preferably, 10 amino acids are identical to the first 10 amino acids of SEQ ID NO: 34 at corresponding positions.

Preferably, the variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu,
b) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu,
c) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu, and wherein the amino acids of the last three positions of the variant are Ala, Leu and Ala,
e) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu,
f) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids of the last three positions of the variant are Ala, Leu and Ala,
g) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids at the three last positions of the variant are Ala, Leu, Ala,
h) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Ala,
i) a variant of SEQ ID NO: 34 of between 15 to 23 amino acids, wherein the amino acid at position 1 is Met, the amino acid at position 2 is Val and wherein a contiguous stretch of 10 amino acids comprises at least 4 Ala and 1 Leu,
j) a variant of SEQ ID NO: 34 of between 15 to 23 amino acids, wherein the amino acid at position 1 is Met, the amino acid at position 2 is Val and wherein a contiguous stretch of 10 amino acids comprises at least 3 Ala and 2 Leu,
k) a variant of SEQ ID NO: 34 of between 15 to 23 amino acids, wherein the amino acid at position 1 is Met, the amino acid at position 2 is Val and wherein a contiguous stretch of 10 amino acids comprises at least 2 Ala and 3 Leu,
l) a variant of SEQ ID NO: 34 of between 15 to 23 amino acids, wherein the amino acid at position 1 is Met, the amino acid at position 2 is Val and wherein a contiguous stretch of 10 amino acids comprises at least 1 Ala and 4 Leu,
m) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Leu,
n) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu,
o) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu, and wherein the amino acids at the three last positions of the variant are Ala, Leu, Ala,
p) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Ala,
q) a variant of SEQ ID NO: 34 of between 15 to 23 amino acids, wherein the amino acid at position 1 is Met, the amino acid at position 2 is Lys and wherein a contiguous stretch of 10 amino acids comprises at least 4 Ala and 1 Leu,
r) a variant of SEQ ID NO: 34 of between 15 to 23 amino acids, wherein the amino acid at position 1 is Met, the amino acid at position 2 is Lys and wherein a contiguous stretch of 10 amino acids comprises at least 3 Ala and 2 Leu,
s) a variant of SEQ ID NO: 34 of between 15 to 23 amino acids, wherein the amino acid at position 1 is Met, the amino acid at position 2 is Lys and wherein a contiguous stretch of 10 amino acids comprises at least 2 Ala and 3 Leu,
t) a variant of SEQ ID NO: 34 of between 15 to 23 amino acids, wherein the amino acid at position 1 is Met, the amino acid at position 2 is Lys and wherein a contiguous stretch of 10 amino acids comprises at least 1 Ala and 4 Leu,
u) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Leu.

In variants (a) to (d) of SEQ ID NO: 25, (a) and (b) of SEQ ID NO: 39, (a) and (b) of SEQ ID NO: 44 and of (a) to (u) of SEQ ID NO: 34 here above, the contiguous stretch is preferably 10 amino acids, more preferably 9 amino acids, even more preferably 8 amino acids and most preferably 7 amino acids.

In variants (a) to (d) of SEQ ID NO: 25, (a) and (b) of SEQ ID NO: 39, (a) and (b) of SEQ ID NO: 44 and of (a) to (u) of SEQ ID NO: 34 here above the contiguous stretch of amino acids comprises preferably at least 5 amino acids selected from Ala or Leu, more preferably at least 6 amino acids selected from Ala or Leu and most preferably at least 7 amino acids selected from Ala or Leu.

Variants of SEQ ID NO: 25, SEQ ID NO: 39, SEQ ID NO: 44, or SEQ ID NO: 34 of between 15 to 23 amino acids may comprise 15 amino acids, 16 amino acids, 17 amino acids, 18 amino acids, 19 amino acids, 20 amino acids, 21 amino acids, 22 amino acids or 23 amino acids.

In a sixth aspect, the present invention relates to the use of a polynucleotide encoding a signal peptide according to the invention for the production of a recombinant polypeptide of interest. Accordingly, the signal peptide is preferably selected from the group consisting of:
a) SEQ ID NO: 25,
b) a variant of SEQ ID NO: 25 of between 15 and 23 amino acids, wherein at least 8 amino acids are identical to the first 10 amino acids of SEQ ID NO: 25 at corresponding positions,
c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
d) SEQ ID NO: 39,
e) a variant of SEQ ID NO: 39 of between 15 and 23 amino acids, wherein at least 8 amino acids are identical to the first 10 amino acids of SEQ ID NO: 39 at corresponding positions,
f) a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
g) SEQ ID NO: 44,
h) a variant of SEQ ID NO: 44 of between 15 and 23 amino acids, wherein at least 8 amino acids are identical to the first 10 amino acids of SEQ ID NO: 44 at corresponding positions,
i) a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
j) SEQ ID NO: 34,
k) a variant of SEQ ID NO: 34 of between 15 and 23 amino acids, wherein at least 8 amino acids are identical to the first 10 amino acids of SEQ ID NO: 34 at corresponding positions,
l) a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to an embodiment, the signal peptide is SEQ ID NO: 25.

According to another embodiment, in the intermediate product, the signal peptide is a variant of SEQ ID NO: 25 of between 15 and 23 amino acids, wherein at least 8 amino acids are identical to the first 10 amino acids of SEQ ID NO: 25 at corresponding positions.

According to another embodiment, the signal peptide is a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, the signal peptide is SEQ ID NO: 39.

According to another embodiment, the signal peptide is a variant of SEQ ID NO: 39 of between 15 and 23 amino acids, wherein at least 8 amino acids are identical to the first 10 amino acids of SEQ ID NO: 39 at corresponding positions.

According to another embodiment, the signal peptide is a variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, the signal peptide is SEQ ID NO: 44.

According to another embodiment, the signal peptide is a variant of SEQ ID NO: 44 of between 15 and 23 amino acids, wherein at least 8 amino acids are identical to the first 10 amino acids of SEQ ID NO: 44 at corresponding positions.

According to another embodiment, the signal peptide is a variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

According to another embodiment, the signal peptide is SEQ ID NO: 34.

According to another embodiment, the signal peptide is a variant of SEQ ID NO: 34 of between 15 and 23 amino acids, wherein at least 8 amino acids are identical to the first 10 amino acids of SEQ ID NO: 34 at corresponding positions.

According to another embodiment, the signal peptide is a variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu.

Preferably, when the signal peptide is
(a): SEQ ID NO: 25,
(b): a variant of SEQ ID NO: 25 of between 15 and 23 amino acids, wherein at least 8 amino acids are identical to the first 10 amino acids of SEQ ID NO: 25 at corresponding positions, or
(c) a variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
   the polypeptide of interest is not a pectin methyl esterase, more preferably the polypeptide of interest is not a pectin methyl esterase from Erwinia chrysanthemi. More preferably, when the signal peptide is (b) or (c), the polypeptide of interest is not a pectin methyl esterase, even more preferably the polypeptide of interest is not a pectin methyl esterase from Erwinia chrysanthemi.

Preferably, the first polynucleotide when encoding SEQ ID NO: 25, is a polynucleotide according to SEQ ID NO: 29. Preferably, the first polynucleotide when encoding SEQ ID NO: 39, is a polynucleotide according to SEQ ID NO: 38. Preferably, the first polynucleotide when encoding SEQ ID NO: 44, is a polynucleotide according to SEQ ID NO: 43. Preferably, the first polynucleotide when encoding SEQ ID NO: 34, is a polynucleotide according to SEQ ID NO: 33.

Preferably, the variant of SEQ ID NO: 25 of between 15 and 23 amino acids, wherein at least 8 amino acids are identical to the first 10 amino acids of SEQ ID NO: 25 at corresponding positions, is a variant wherein at least 9 amino acids are identical to the first 10 amino acids of SEQ ID NO: 25 at corresponding positions. More preferably, 10 amino acids are identical to the first 10 amino acids of SEQ ID NO: 25 at corresponding positions.

Preferably, the variant of SEQ ID NO: 25 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu,
c) a variant of SEQ ID NO: 25 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 25 of between 15 to 23 amino acids, wherein the amino acid at position 1 is Met, the amino acid at position 2 is Val and the amino acid at position 3 is Lys and wherein a contiguous stretch of 10 amino acids comprises at least 5 amino acids selected from Ala or Leu, and wherein the amino acids of the last three positions of the variant are Ala, Leu and Ala.

Preferably, the variant of SEQ ID NO: 39 of between 15 and 23 amino acids, wherein at least 8 amino acids are identical to the first 10 amino acids of SEQ ID NO: 39 at corresponding positions, is a variant wherein at least 9 amino acids are identical to the first 10 amino acids of SEQ ID NO: 39 at corresponding positions. More preferably, 10 amino acids are identical to the first 10 amino acids of SEQ ID NO: 39 at corresponding positions.

Preferably, the variant of SEQ ID NO: 39 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 39 of between 15 to 23 amino acids, wherein the amino acid at position 1 is Met, the amino acid at position 2, 3 and/or 5 is Val or Lys and wherein a contiguous stretch of 10 amino acids comprises at least 5 amino acids selected from Ala or Leu,
b) a variant of SEQ ID NO: 39 of between 15 to 23 amino acids, wherein the amino acid at position 1 is Met, the amino acid at position 2 is Val and the amino acid at position 5 is Lys and wherein a contiguous stretch of 10 amino acids comprises at least 5 amino acids selected from Ala or Leu.

Preferably, the variant of SEQ ID NO: 44 of between 15 and 23 amino acids, wherein at least 8 amino acids are identical to the first 10 amino acids of SEQ ID NO: 44 at corresponding positions, is a variant wherein at least 9 amino acids are identical to the first 10 amino acids of SEQ ID NO: 44 at corresponding positions. More preferably, 10 amino acids are identical to the first 10 amino acids of SEQ ID NO: 44 at corresponding positions.

Preferably, the variant of SEQ ID NO: 44 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 44 of between 15 to 23 amino acids, wherein the amino acid at position 1 is Met, the amino acid at position 2, 3 and/or 4 is Val or Lys and wherein a contiguous stretch of 10 amino acids comprises at least 5 amino acids selected from Ala or Leu,
b) a variant of SEQ ID NO: 44 of between 15 to 23 amino acids, wherein the amino acid at position 1 is Met, the amino acid at position 2 is Val and the amino acid at position 4 is Lys and wherein a contiguous stretch of 10 amino acids comprises at least 5 amino acids selected from Ala or Leu.

Preferably, the variant of SEQ ID NO: 34 of between 15 and 23 amino acids, wherein at least 8 amino acids are identical to the first 10 amino acids of SEQ ID NO: 34 at corresponding positions, is a variant wherein at least 9 amino acids are identical to the first 10 amino acids of SEQ ID NO: 34 at corresponding positions. More preferably, 10 amino acids are identical to the first 10 amino acids of SEQ ID NO: 34 at corresponding positions.

Preferably, the variant of SEQ ID NO: 34 of between 15 to 23 amino acids, wherein the amino acid at position 1 is Met and the amino acid at position 2 is Val or Lys and wherein a contiguous stretch of 10 amino acids comprises at least 5 amino acids selected from Ala or Leu, is one selected from the group of:
a) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu,
b) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu,
c) a variant of SEQ ID NO: 34 of between 15 to 23 amino acids, wherein the amino acid at position 1 is Met, the amino acid at position 2, 3 and/or 4 is Lys and wherein a contiguous stretch of 10 amino acids comprises at least 5 amino acids selected from Ala or Leu, and wherein the amino acids of the last three positions of the variant are Ala, Leu and Ala,
d) a variant of SEQ ID NO: 34 of between 15 to 23 amino acids, wherein the amino acid at position 1 is Met, the amino acid at position 2 is Lys and the amino acid at position 3 and/or 4 is Lys and wherein a contiguous stretch of 10 amino acids comprises at least 5 amino acids selected from Ala or Leu, and wherein the amino acids of the last three positions of the variant are Ala, Leu and Ala,
e) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu,
f) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids of the last three positions of the variant are Ala, Leu and Ala,
g) a variant of SEQ ID NO: 34 of between 15 to 23 amino acids, wherein the amino acid at position 1 is Met, the amino acid at position 2 is Val and wherein a contiguous stretch of 10 amino acids comprises at least 5 amino acids selected from Ala or Leu, and wherein the amino acids at the three last positions of the variant are Ala, Leu, Ala,
h) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Ala,
i) a variant of SEQ ID NO: 34 of between 15 to 23 amino acids, wherein the amino acid at position 1 is Met, the amino acid at position 2 is Val and wherein a contiguous stretch of 10 amino acids comprises at least 4 Ala and 1 Leu,
j) a variant of SEQ ID NO: 34 of between 15 to 23 amino acids, wherein the amino acid at position 1 is Met, the amino acid at position 2 is Val and wherein a contiguous stretch of 10 amino acids comprises at least 3 Ala and 2 Leu,
k) a variant of SEQ ID NO: 34 of between 15 to 23 amino acids, wherein the amino acid at position 1 is Met, the amino acid at position 2 is Val and wherein a contiguous stretch of 10 amino acids comprises at least 2 Ala and 3 Leu,
l) a variant of SEQ ID NO: 34 of between 15 to 23 amino acids, wherein the amino acid at position 1 is Met, the amino acid at position 2 is Val and wherein a contiguous stretch of 10 amino acids comprises at least 1 Ala and 4 Leu,
m) a variant of SEQ ID NO: 34 of between 15 to 23 amino acids, wherein the amino acid at position 1 is Met, the amino acid at position 2 is Val and wherein a contiguous stretch of 10 amino acids comprises at least 5 Leu,
n) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu,
o) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 amino acids selected from Ala or Leu, and wherein the amino acids at the three last positions of the variant are Ala, Leu, Ala,
p) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Ala,
q) a variant of SEQ ID NO: 34 of between 15 to 23 amino acids, wherein the amino acid at position 1 is Met, the amino acid at position 2 is Lys and wherein a contiguous stretch of 10 amino acids comprises at least 4 Ala and 1 Leu,
r) a variant of SEQ ID NO: 34 of between 15 to 23 amino acids, wherein the amino acid at position 1 is Met, the amino acid at position 2 is Lys and wherein a contiguous stretch of 10 amino acids comprises at least 3 Ala and 2 Leu,
s) a variant of SEQ ID NO: 34 of between 15 to 23 amino acids, wherein the amino acid at position 1 is Met, the amino acid at position 2 is Lys and wherein a contiguous stretch of 10 amino acids comprises at least 2 Ala and 3 Leu,
t) a variant of SEQ ID NO: 34 of between 15 to 23 amino acids, wherein the amino acid at position 1 is Met, the amino acid at position 2 is Lys and wherein a contiguous stretch of 10 amino acids comprises at least 1 Ala and 4 Leu,
u) a variant of SEQ ID NO: 34 of between 15 to 23 amino acids, wherein the amino acid at position 1 is Met, the amino acid at position 2 is Lys and wherein a contiguous stretch of 10 amino acids comprises at least 5 Leu.

In variants (a) to (d) of SEQ ID NO: 25, (a) and (b) of SEQ ID NO: 39, (a) and (b) of SEQ ID NO: 44 and of (a) to (u) of SEQ ID NO: 34 here above, the contiguous stretch is preferably 10 amino acids, more preferably 9 amino acids, even more preferably 8 amino acids and most preferably 7 amino acids.

In variants (a) to (d) of SEQ ID NO: 25, (a) and (b) of SEQ ID NO: 39, (a) and (b) of SEQ ID NO: 44 and of (a) to (u) of SEQ ID NO: 34 here above the contiguous stretch of amino acids comprises preferably at least 5 amino acids selected from Ala or Leu, more preferably at least 6 amino acids selected from Ala or Leu and most preferably at least 7 amino acids selected from Ala or Leu.

Variants of SEQ ID NO: 25, SEQ ID NO: 39, SEQ ID NO: 44, or SEQ ID NO: 34 of between 15 to 23 amino acids may comprise 15 amino acids, 16 amino acids, 17 amino acids, 18 amino acids, 19 amino acids, 20 amino acids, 21 amino acids, 22 amino acids or 23 amino acids.

The sequence information as provided herein should not be so narrowly construed as to require inclusion of erroneously identified bases. The specific sequences disclosed herein can be readily used to isolate the complete gene from the respective host cells which in turn can easily be subjected to further sequence analyses thereby identifying sequencing errors.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a nucleic acid sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

The invention described and claimed herein is not to be limited in scope by the specific embodiments herein enclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In case of conflict, the present disclosure including definitions will be taken as a guide.

The present invention is further illustrated by the following examples.

### EXAMPLES

### Strains

WT 1: This A. niger strain is used as a wild-type strain. This strain is deposited at the CBS Institute under the deposit number CBS 513.88.

WT 2: This A. niger strain is a WT 1 strain comprising a deletion of the gene encoding glucoamylase (glaA). WT 2 was constructed by using the “MARKER-GENE FREE” approach as described in EP 0 635 574 B1. In this patent it is extensively described how to delete glaA specific DNA sequences in the genome of CBS 513.88. The procedure resulted in a MARKER-GENE FREE ΔglaA recombinant A. niger CBS 513.88 strain, possessing finally no foreign DNA sequences at all.

WT 3: This A. niger strain is a WT 2 strain comprising a deletion which results in an oxalate deficient A. niger strain. WT 3 was constructed by using the method as described in EP1157100 and U.S. Pat. No. 6,936,438, in which an oxalate deficient strain was obtained by deletion of the oahA gene, encoding oxaloacetate hydrolase, Strain WT 3 was selected as a representative strain with the oahA gene inactivated in the WT 2 strain background.

Alternatively, in EP1590444 it is extensively described how to screen for an oxalate deficient mutant A. niger strain. Following the examples 1 and 2 of EP1590444, it is described how an oxalate deficient mutant strain of WT 2 can be obtained.

WT 4: This A. niger strain is a WT 3 strain comprising the deletion of three genes encoding alpha-amylases (amyB, amyBI and amyBII) in three subsequent steps. The construction of deletion vectors and genomic deletion of these three genes has been described in detail in WO2005095624. The vectors pDEL-AMYA, pDEL-AMYBI and pDEL-AMYBII, described in WO2005095624, have been used according the “MARKER-GENE FREE” approach as described in EP 0 635 574 B1. The procedure described above resulted in an oxalate deficient, MARKER-GENE FREE ΔglaA, ΔamyA, ΔamyBI and ΔamyBII amylase-negative recombinant A. niger CBS 513.88 strain, possessing finally no foreign DNA sequences at all. As such, WT 4 has a low amylase background and is more optimized for alpha-amylase expression and expression detection compared to WT 1.

### Molecular Biology Techniques

In these strains, using molecular biology techniques known to the skilled person (see: Sambrook & Russell, Molecular Cloning: A Laboratory Manual, 3rd Ed., CSHL Press, Cold Spring Harbor, N.Y., 2001), several genes were over expressed and others were down regulated as described below. Examples of the general design of expression vectors for gene over expression and disruption vectors for down-regulation, transformation, use of markers and selective media can be found in WO199846772, WO199932617, WO2001121779, WO2005095624, EP 635574B and WO2005100573.

A. niger Shake Flask Fermentations

A. niger strains are precultured in 20 ml preculture medium as described in the Examples: “Aspergillus niger shake flask fermentations” section of WO 99/32617. After overnight growth, 10 ml of this culture is transferred to Fermentation Medium (FM).

Fermentation medium (FM) contains per liter: 82.5 g Glucose.1H2O, 25 g Maldex 15 (Boom Meppel, Netherlands), 2 g Citric acid, 4.5 g NaH2PO4.1H2O, 9 g KH2PO4, 15 g (NH4)2SO4, 0.02 g ZnCl2, 0.1 g MnSO4.1H2O, 0.015 g CuSO4.5H2O, 0.015 g CoCl2.6H2O, 1 g MgSO4.7H2O, 0.1 g CaCl2.2H2O, 0.3 g FeSO4.7H2O, 30 g MES (2-[N-Morpholino]ethanesulfonic acid), pH=6.

Fermentation in FM is performed in 500 ml flasks with baffle with 100 ml fermentation broth at 34° C. and 170 rpm for the number of days indicated, generally as described in WO99/32617.

### Fungal Alpha-Amylase Activity

To determine the alpha-amylase activity in A. niger culture broth, the Megazyme cereal alpha-amylase kit is used (Megazyme, CERALPHA alpha amylase assay kit, catalogue. ref. K-CERA, year 2000-2001), according protocol of the supplier. The measured activity is based on hydrolysis of non-reducing-end blocked ρ-nitrophenyl maltoheptaoside in the presence of excess glucoamylase and α-glucosidase. The amount of formed ρ-nitrophenol is a measure for alpha-amylase activity present in a sample.

### Glucose Oxidase Activity

To determine the glucose oxidase activity in A. niger culture broth, glucose oxidase was measured spectrophotometrically at 450 nm using o-dianisidine as described by Witteveen et al. 1990 (“Glucose oxidase overproducing and negative mutants of Aspergillus niger”, Appl. Microbiol. Biotechnol 33:683-686).

### Example 1

### Construction of Modified Aspergillus Expression Constructs for A. niger Alpha-Amylase AmyB and P. chrysogenum Glucose Oxidase goxA

The DNA sequence of the amyB gene encoding the alpha-amylase protein can be retrieved from EMBL Nucleotide Sequence Database (http://www.ebi.ac.uk/embl/index.html) under accession numbers XM_{—}001395712.1, XM_{—}001390741.1 or CAK46324. The genomic sequence of the native A. niger amyB gene is shown as SEQ ID NO. 1. The corresponding coding or cDNA sequence of amyB is shown as SEQ ID NO. 2. The translated sequence of SEQ ID NO. 2 is assigned as the SEQ ID NO. 3, representing the A. niger alpha-amylase protein AmyB. This sequence has also a 100% similarity with the A. oryzae alpha-amylase protein (Wirsel S., Lachmund A., Wildhardt G., Ruttkowski E., “Three alpha-amylase genes of Aspergillus oryzae exhibit identical intron-exon organization” (1989) Mol. Microbiol. 3:3-14). The native secreted A. niger mature alpha-amylase peptide is assigned as the SEQ ID NO. 4. Optimization according a method of the invention has been performed with an optimised amyB cDNA sequence and improved expression vectors as detailed below.

For expression analysis in Aspergillus species of variants of A. niger amyB constructs, the amyB coding sequence comprised a codon optimized (CO) coding sequence for the alpha-amylase encoding amyB gene (as described in detail in WO2008/000632). Both the strong A. niger glucoamylase glaA promoter and the alpha-amylase amyB promoter were applied for over-expression of the alpha amylase enzyme in A. niger using pGBFIN-based expression constructs (as described in WO1999/32617 and WO2006/077258). The translational initiation sequences of the glucoamylase glaA and alpha-amylase amyB promoter have been modified into 5′-CACCGTCAAA ATG-3′ in all subsequent amyB expression constructs generated (as also detailed in WO2006/077258). The BstX1 site (5′-CCANNNNN/NTGG-3′), present in the native alpha-amylase amyB promoter, was removed in some vectors to facilitate cloning of signal sequence variants. In addition, an optimal translational termination sequence was used, and therefore the wild-type amyB 5′-TGA-3′ translational termination sequence was replaced by 5′-TAAA-3′ (as detailed in WO2006/077258) in all expression constructs.

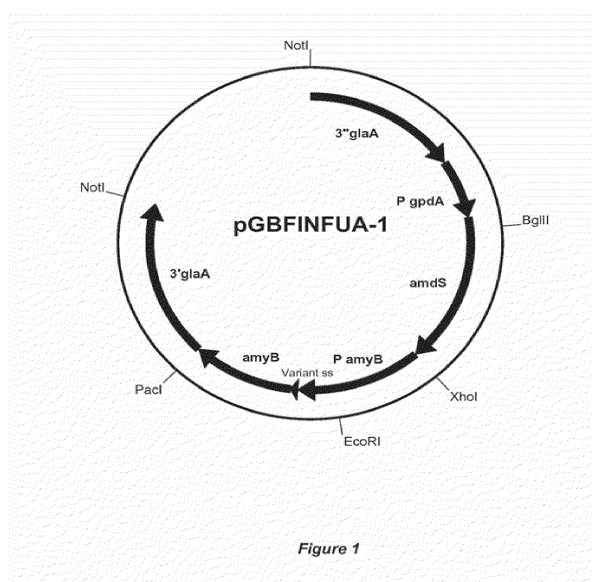

Appropriate restriction sites were introduced at both ends to allow cloning in an expression vector. At the 5′-end an XhoI site was introduced and at the 3′-end a PacI site. The DNA fragment of the reference constructs comprising a modified genomic glaA or amyB promoter and optimized amyB cDNA sequence was synthesized completely, subcloned, and sequence verified by sequence analysis. The XhoI-PacI restriction sites at the ends of the two synthesized fragments were used to allow cloning in the large vector fragment of an XhoI and PacI digested pGBFINFUA-1 expression vector (the pGBFINFUA-1 vector is also described in WO2006/077258 and WO2008/000632, see FIG. 1 for general layout of the vector), generating pGBFINFUA-6 and pGBFINFUA-3, respectively.

All DNA fragments of the modified AmyB sequences, which vary a.o. in signal sequences according a method of the invention, were designed, synthesized completely as EcoRI-PacI or EcoRI-BstX1 fragments, subcloned and sequence verified. The EcoRI-PacI/BstX1 restriction sites at the ends of all synthesized fragments were used to allow cloning in the large vector fragment of EcoRI and PacI/BstX1 digested pGBFINFUA-3 or EcoRI and PacI digested pGBFINFUA-6 expression vectors, generating variant pGBFINFUA-expression vectors. After sequence verification of the respective vectors, the variant expression constructs were named as described below in Table 1 and 2. All characteristics and reference to respective sequences of all pGBFINFUA-constructs can be deduced from Table 1 and Table 2.

TABLE 1
Modified expression constructs for alpha-amylase AmyB expression in A. niger under
control of the glaA promoter

| | | | | | | | Signal | |
|---|---|---|---|---|---|---|---|---|
| | | | | | | | sequence | |
| | | | | | Coding | | coding | Amino acid |
| | | | | Coding | sequence | Protein | sequence | signal |
| | SEQ | | Signal | sequence | SEQ ID | SEQ ID | SEQ ID | sequence |
| Plasmid name | ID NO | Promoter | sequence type | AmyB | NO | NO | NO | SEQ ID NO |
| pGBFINFUA-6 | 5 | glaA | Fungal amylase | Codon | 2 | 3 | 6 | 7 |
| | | | (amyB) | optimized | | | | |
| | | | | (CO) amyB | | | | |
| | | | | cDNA | | | | |
| pGBFINFUA-8 | 8 | glaA | Fungal amylase | CO amyB | 9 | 3 | 10 | 7 |
| | | | (amyB) | cDNA | | | | |
| | | | Codon | | | | | |
| | | | optimized | | | | | |
| pGBFINFUA-11 | 11 | glaA | Glucoamylase | CO amyB | 12 | 13 | 14 | 15 |
| | | | (glaA) | cDNA | | | | |
| | | | Codon | | | | | |
| | | | optimized | | | | | |
| pGBFINFUA-12 | 21 | glaA | Pectin methyl | CO amyB | 22 | 23 | 24 | 25 |
| | | | esterase | cDNA | | | | |
| | | | (pmeA) | | | | | |
| pGBFINFUA-13 | 26 | glaA | Pectin methyl | CO amyB | 27 | 28 | 29 | 25 |
| | | | esterase | cDNA | | | | |
| | | | (pmeA) | | | | | |
| | | | Codon | | | | | |
| | | | optimized | | | | | |
| pGBFINFUA-15 | 30 | glaA | Optimal | CO amyB | 31 | 32 | 33 | 34 |
| | | | synthetic signal | cDNA | | | | |
| | | | sequence | | | | | |
| | | | Codon | | | | | |
| | | | optimized | | | | | |
| pGBFINFUA-16 | 35 | glaA | Xylanase ss | CO amyB | 36 | 37 | 38 | 39 |
| | | | Codon | cDNA | | | | |
| | | | optimized | | | | | |
| pGBFINFUA-18 | 40 | glaA | Chitinase ss | CO amyB | 41 | 42 | 43 | 44 |
| | | | Codon | cDNA | | | | |
| | | | optimized | | | | | |

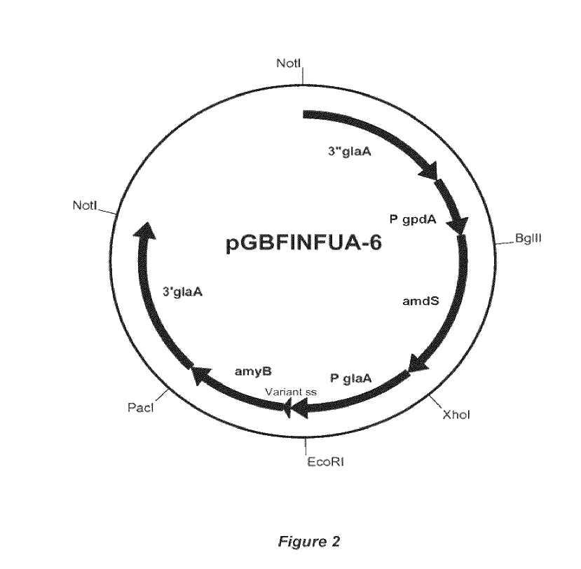

In all Tables of Example 1 herein, the sequences of the EcoRI-PacI part of all pGBFIN plasmids are indicated under “SEQ ID NO”, the full gene coding sequences and the translated sequences of the coding sequences are according to the amino acid sequence as depicted in “Coding sequence SEQ ID NO” and “Protein SEQ ID NO”, respectively, and the nucleotide and translated amino acid sequence of the signal sequence used is indicated under “Signal sequence coding sequence SEQ ID NO” and “Amino acid signal sequence SEQ ID NO”. The general layout of pGBFINFUA-6 and derived vectors can be found in FIG. 2, whereas the layout of vectors pGBFINFUA-1, pGBFINFUA-3 and pGBFINFUA-21 can be found in FIG. 1.

TABLE 2
Modified expression constructs for alpha-amylase AmyB expression in A. niger under
control of the amyB promoter

| | | | | | | | Signal | |
|---|---|---|---|---|---|---|---|---|
| | | | | | | | sequence | |
| | | | | | Coding | | coding | Amino acid |
| | | | | Coding | sequence | Protein | sequence | signal |
| | SEQ | | Signal | sequence | SEQ ID | SEQ ID | SEQ ID | sequence |
| Plasmid name | ID NO | Promoter | sequence type | AmyB | NO | NO | NO | SEQ ID NO |
| pGBFINFUA-3 | 45 | amyB | Fungal amylase | modified | 2 | 3 | 6 | 7 |
| | | | (amyB) | amyB | | | | |
| | | | | cDNA | | | | |
| pGBFINFUA-21 | 46 | amyB | Pectin methyl | modified | 27 | 28 | 29 | 25 |
| | | | esterase | amyB | | | | |
| | | | (pmeA) | cDNA | | | | |
| | | | Codon | | | | | |
| | | | optimized | | | | | |

The DNA sequence of the goxA gene, with gene code Pc20g09560 and encoding the Penicillium chrysogenum glucose oxidase protein, can be retrieved from EMBL Nucleotide Sequence Database (http://www.ebi.ac.uk/embl/index.html) under accession number AM920435.1. The translated sequence of Pc20g09560 is assigned as SEQ ID NO. 49, which is representing the P. chrysogenum glucose oxidase protein GoxA.

Expression of the goxA gene or gene fragments were performed with improved expression vectors as detailed above and optimization according a method of the invention has been performed with a codon-pair optimized goxA cDNA sequence, which can be identified as SEQ ID NO. 48.

The two DNA fragments of the modified GoxA constructs, which vary a.o. in signal sequences according a method of the invention and comprising among others part of the glaA promoter and an optimized GoxA cDNA sequence, were designed, synthesized completely as EcoRI-PacI fragments, subcloned and sequence verified. The EcoRI-PacI restriction sites at the ends of the synthesized fragments were used to allow cloning in the large vector fragment of EcoRI and PacI digested pGBFINFUA-6 expression vector, generating variant pGBFINGOX-expression vectors. After sequence verification of the respective vectors, the variant expression constructs were named as described below in Table 3. All characteristics and reference to respective sequences of the two pGBFINGOX-constructs can be deduced from Table 3.

TABLE 3
Modified expression constructs for P. chrysogenum glucose oxidase expression in A. niger

| | | | | | | | Signal | |
|---|---|---|---|---|---|---|---|---|
| | | | | | | | sequence | |
| | | | | | Coding | | coding | Amino |
| | | | Signal | Coding | sequence | Protein | sequence | acid signal |
| | SEQ | | sequence | sequence | SEQ ID | SEQ ID | SEQ ID | sequence |
| Plasmid name | ID NO | Promoter | type | AmyB | NO | NO | NO | SEQ ID NO |
| pGBFINGOX-1 | 47 | glaA | Glucose | modified | 48 | 49 | 50 | 51 |
| | | | oxidase (goxA) | goxA cDNA | | | | |
| pGBFINGOX-2 | 52 | glaA | Pectin methyl | modified | 53 | 54 | 29 | 25 |
| | | | esterase | goxA cDNA | | | | |
| | | | (pmeA) | | | | | |
| | | | Codon | | | | | |
| | | | optimized | | | | | |

### Example 2

### Expression of Wild-Type and Modified Expression Constructs for A. niger Alpha-Amylase and P. chrysogenum Glucose Oxidase in A. niger

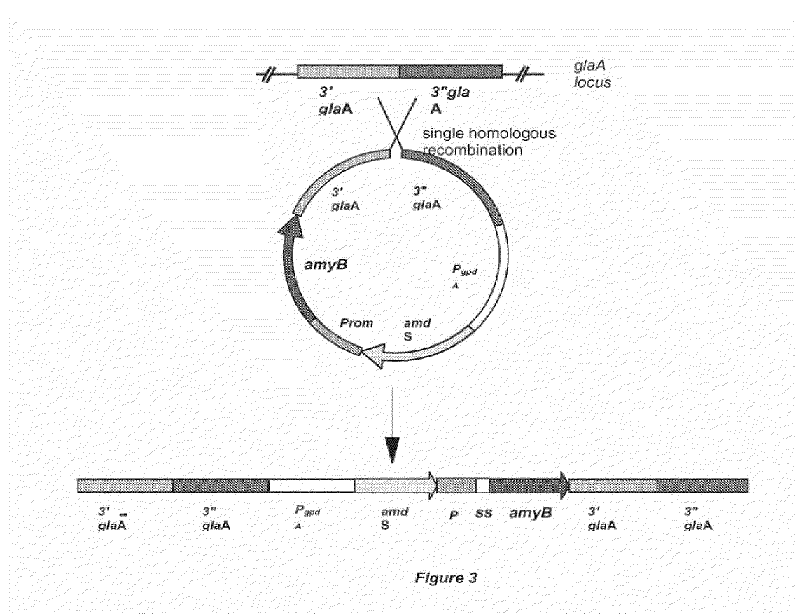

The pGBFINFUA- and pGBFFINGOX-expression constructs, prepared in Example 1 (super), were introduced in A. niger by transformation as described below and according to the strategy depicted in FIG. 3.

In order to introduce the different pGBFINFUA-vectors (Table 1 and 2) and the two different pGBFINGOX-vectors (Table 3) in WT 4, a transformation and subsequent selection of transformants was carried out as described in WO1998/46772 and WO1999/32617. In brief, linear DNA of the pGBFINFUA- and pGBFINGOX-constructs was isolated and used to transform A. niger WT4. Transformants were selected on acetamide media and colony purified according standard procedures. Colonies were diagnosed for integration at the glaA locus and for copy number using PCR. Three independent transformants of each pGBFINFUA-construct with similar estimated copy numbers (putative single copy) were selected and named using the number of the transforming plasmid, as for example FUA-3-1, FUA-3-2, FUA-3-3, FUA-6-1, etc. . . . , respectively.

Similarly, five independent transformants of each pGBFINGOX-construct with similar estimated copy numbers (putative single copy) were selected and named using the number of the transforming plasmid, as for example GOX-1-1, GOX-1-2, GOX-1-3, . . . , GOX-2-1, GOX-2-2, GOX-2-3, etc. . . . , respectively.

The selected FUA- and GOX-strains and A. niger WT 1 and WT 4 were used to perform shake flask experiments in 100 ml of the medium as described above at 34° C. and 170 rpm in an incubator shaker using a 500 ml baffled shake flask. After day 3 and day 4 or day 4 and day 5 of fermentation, samples were taken to determine alpha-amylase activities or glucose oxidase activities, respectively.

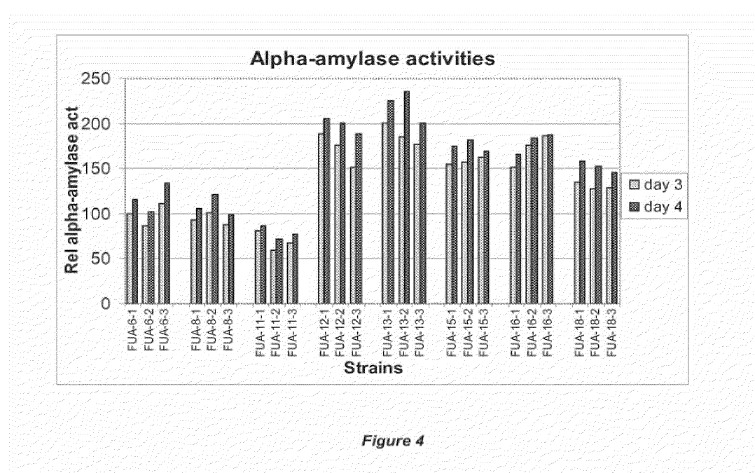

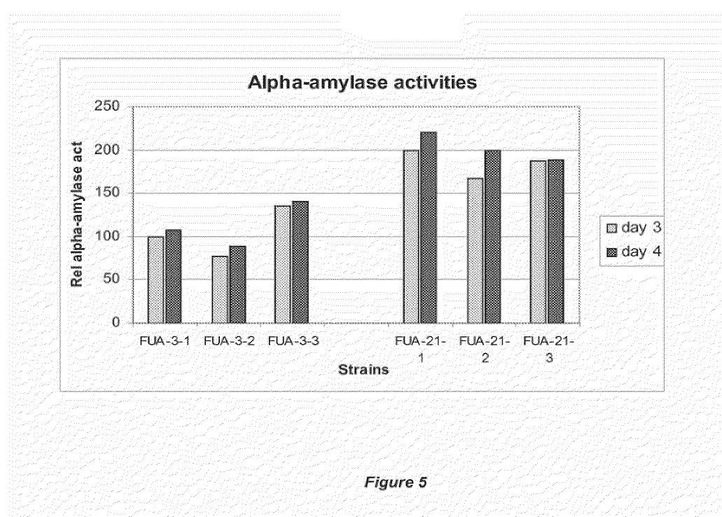

The production of alpha-amylase produced by the transformants of the different A. niger FUA-transformants containing the different constructs, was measured in the culture supernatant. Use of an endogenous amyB signal sequence, with or without codon-pair optimization or use of an optimized glucoamylase signal sequence, no positive effect on alpha-amylase production and expression could be found. Surprisingly, a clear positive effect of the use of a modified and optimal signal sequence of the invention on alpha-amylase production was observed when using the glucoamylase promoter, as can be learned from FIG. 4. Multiple optimal signal sequences of the invention give a positive effect on the production of alpha-amylase with the pectin methyl esterase (i.e. pmeA in pGBFINFUA-12/13) being the best. In FIG. 5, also a clear positive effect of the use of a pmeA signal sequence of the invention on alpha-amylase production was observed in combination with the alpha-amylase amyB promoter.

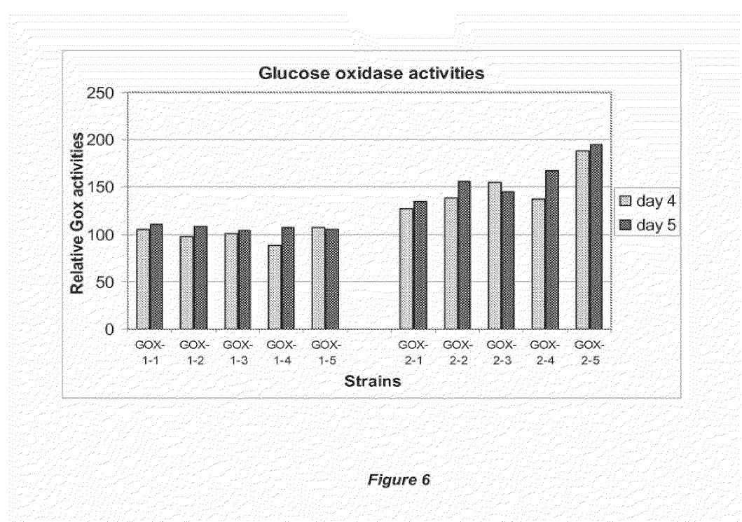

The production of P. chrysogenum glucose oxidase GoxA was measured in five transformants of two different A. niger GOX-transformants. Also here, a clear positive effect of the use of a signal sequence of the invention (i.e. pmeA) on glucose oxidase production was observed, as can be learned from FIG. 6.

Thus, a positive effect for the use of an optimal signal sequence and more specifically the pmeA signal sequence according a method of the invention was found in combination with the strong alpha-amylase amyB and glucoamylase glaA promoter. Also, the pmeA signal sequence according a method of the invention fused to the goxA glucose oxidase encoding enzyme resulted in a clearly increased extracellular GoxA enzyme production. Additionally, positive effects of combinations of a method of the invention with a modified translation initiation site, a codon optimized coding sequence and/or a translational termination sequence on alpha-amylase production, were observed. These results indicate clearly an additive effect of a modification the invention with other sequence optimizations identified for expression constructs.

Clearly, these examples show how a method of the invention, for example a pmeA signal sequence fused to the native alpha-amylase or glucose oxidase sequence, can be used for improved secretion and production of alpha-amylase or glucose oxidase in A. niger or any other protein of interest in a filamentous fungus. Additionally, these results indicate that the method of the invention can be broadly applied to improve protein expression in a host, although the expression construct and host has already several other optimizations, such as for example a strong promoter, an improved translation initiation sequence, an improved translational termination sequence, an optimized codon and codon pair usage and/or an improved host for protein expression.

1. A process for the production of a recombinant polypeptide of interest comprising:
(i) cultivation of a filamentous fungal host cell under conditions conducive to the production of said polypeptide, said filamentous fungal host cell comprising a first polynucleotide linked in translational reading frame to a second polynucleotide, said second polynucleotide encoding a polypeptide of interest, said first polynucleotide encoding a signal peptide selected from the group consisting of:
a) SEQ ID NO: 25,
b) a variant of SEQ ID NO: 25 of from 15 to 23 amino acids, wherein at least 8 amino acids are identical to the first 10 amino acids of SEQ ID NO: 25 at corresponding positions,
c) a variant of SEQ ID NO: 25 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
d) SEQ ID NO: 39,
e) a variant of SEQ ID NO: 39 of from 15 to 23 amino acids, wherein at least 8 amino acids are identical to the first 10 amino acids of SEQ ID NO: 39 at corresponding positions,
f) a variant of SEQ ID NO: 39 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu
g) SEQ ID NO: 44,
h) a variant of SEQ ID NO: 44 of from 15 to 23 amino acids, wherein at least 8 amino acids are identical to the first 10 amino acids of SEQ ID NO: 44 at corresponding positions,
i) a variant of SEQ ID NO: 44 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu
j) SEQ ID NO: 34,
k) a variant of SEQ ID NO: 34 of from 15 to 23 amino acids, wherein at least 8 amino acids are identical to the first 10 amino acids of SEQ ID NO: 34 at corresponding positions, and
l) a variant of SEQ ID NO: 34 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
(ii) and optionally, isolation of said polypeptide from the culture medium, with the proviso that when the signal peptide is (b) or (c), the polypeptide of interest is not a pectin methyl esterase from Erwinia chrysanthemi.

2. The polypeptide obtained by the process according to claim 1.

3. A recombinant polypeptide encoded by a first polynucleotide linked in translational reading frame to a second polynucleotide, said second polynucleotide encoding a polypeptide of interest, said first polynucleotide encoding a signal peptide selected from the group consisting of:
a) SEQ ID NO: 25,
b) a variant of SEQ ID NO: 25 of from 15 to 23 amino acids, wherein at least 8 amino acids are identical to the first 10 amino acids of SEQ ID NO: 25 at corresponding positions,
c) a variant of SEQ ID NO: 25 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
d) SEQ ID NO: 39,
e) a variant of SEQ ID NO: 39 of from 15 to 23 amino acids, wherein at least 8 amino acids are identical to the first 10 amino acids of SEQ ID NO: 39 at corresponding positions,
f) a variant of SEQ ID NO: 39 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
g) SEQ ID NO: 44,
h) a variant of SEQ ID NO: 44 of from 15 to 23 amino acids, wherein at least 8 amino acids are identical to the first 10 amino acids of SEQ ID NO: 44 at corresponding positions,
i) a variant of SEQ ID NO: 44 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
j) SEQ ID NO: 34,
k) a variant of SEQ ID NO: 34 of from 15 to 23 amino acids, wherein at least 8 amino acids are identical to the first 10 amino acids of SEQ ID NO: 34 at corresponding positions, and
l) a variant of SEQ ID NO: 34 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
with the proviso that when the signal peptide is (b) or (c), the polypeptide of interest is not a pectin methyl esterase from Erwinia chrysanthemi.

4. A recombinant expression construct comprising: a first polynucleotide linked in translational reading frame to a second polynucleotide, said second polynucleotide encoding a polypeptide of interest, said first polynucleotide encoding a signal peptide selected from the group consisting of:
a) SEQ ID NO: 25,
b) a variant of SEQ ID NO: 25 of from 15 to 23 amino acids, wherein at least 8 amino acids are identical to the first 10 amino acids of SEQ ID NO: 25 at corresponding positions,
c) a variant of SEQ ID NO: 25 of from 15 to 23 amino acids, wherein the amino acid at position 1 is Met and the amino acid at position 2 is Val or Lys and
wherein a contiguous stretch of 10 amino acids comprises at least 5 amino acids selected from Ala or Leu,
d) SEQ ID NO: 39,
e) a variant of SEQ ID NO: 39 of from 15 to 23 amino acids, wherein at least 8 amino acids are identical to the first 10 amino acids of SEQ ID NO: 39 at corresponding positions,
f) a variant of SEQ ID NO: 39 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
g) SEQ ID NO: 44,
h) a variant of SEQ ID NO: 44 of from 15 to 23 amino acids, wherein at least 8 amino acids are identical to the first 10 amino acids of SEQ ID NO: 44 at corresponding positions,
i) a variant of SEQ ID NO: 44 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
j) SEQ ID NO: 34,
k) a variant of SEQ ID NO: 34 of from 15 to 23 amino acids, wherein at least 8 amino acids are identical to the first 10 amino acids of SEQ ID NO: 34 at corresponding positions, and
l) a variant of SEQ ID NO: 34 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu, with the proviso that when the signal peptide is (b) or (c), the polypeptide of interest is not a pectin methyl esterase from Erwinia chrysanthemi.

5. The recombinant polypeptide according to claim 3, further comprising a promoter operably linked to said first and second polynucleotide.

6. A recombinant expression vector comprising the expression construct according to claim 4.

7. A recombinant filamentous fungal host cell comprising the expression construct according to claim 4.

8. A signal peptide selected from the group consisting of:
a) SEQ ID NO: 25,
b) a variant of SEQ ID NO: 25 of from 15 to 23 amino acids, wherein at least 8 amino acids are identical to the first 10 amino acids of SEQ ID NO: 25 at corresponding positions,
c) a variant of SEQ ID NO: 25 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
d) SEQ ID NO: 39,
e) a variant of SEQ ID NO: 39 of from 15 to 23 amino acids, wherein at east 8 amino acids are identical to the first 10 amino acids of SEQ ID NO: 39 at corresponding positions,
f) a variant of SEQ ID NO: 39 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
g) SEQ ID NO: 44,
h) a variant of SEQ ID NO: 44 of from 15 to 23 amino acids, wherein at least 8 amino acids are identical to the first 10 amino acids of SEQ ID NO: 44 at corresponding positions,
i) a variant of SEQ ID NO: 44 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
j) SEQ ID NO: 34,
k) a variant of SEQ ID NO: 34 of from 15 to 23 amino acids, wherein at least 8 amino acids are identical to the first 10 amino acids of SEQ ID NO: 34 at corresponding positions, and
l) a variant of SEQ ID NO: 34 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
which is suitable for the production of a recombinant polypeptide of interest, with the proviso that when the signal peptide is (b) or (c), the polypeptide of interest is not a pectin methyl esterase from Erwinia chrysanthemi.

9. A polynucleotide encoding a signal peptide selected from the group consisting of:
a) SEQ ID NO: 25,
b) a variant of SEQ ID NO: 25 of from 15 to 23 amino acids, wherein at least 8 amino acids are identical to the first 10 amino acids of SEQ ID NO: 25 at corresponding positions,
c) a variant of SEQ ID NO: 25 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
d) SEQ ID NO: 39,
e) a variant of SEQ ID NO: 39 of from 15 to 23 amino acids, wherein at least 8 amino acids are identical to the first 10 amino acids of SEQ ID NO: 39 at corresponding positions,
f) a variant of SEQ ID NO: 39 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
g) SEQ ID NO: 44,
h) a variant of SEQ ID NO: 44 of from 15 to 23 amino acids, wherein at least 8 amino acids are identical to the first 10 amino acids of SEQ ID NO: 44 at corresponding positions,
i) a variant of SEQ ID NO: 44 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
j) SEQ ID NO: 34,
k) a variant of SEQ ID NO: 34 of from 15 to 23 amino acids, wherein at least 8 amino acids are identical to the first 10 amino acids of SEQ ID NO: 34 at corresponding positions, and
l) a variant of SEQ ID NO: 34 of from 15 to 23 amino acids, wherein the amino acid at position 1 is Met and the amino acid at position 2 is Val or Lys and wherein a contiguous stretch of 10 amino acids comprises at least 5 amino acids selected from Ala or Leu,
which is suitable for the production of a recombinant polypeptide of interest, with the proviso that when the signal peptide is (b) or (c), the polypeptide of interest is not a pectin methyl esterase from Erwinia chrysanthemi.

10. A recombinant expression vector comprising the expression construct according to claim 5.

11. A recombinant filamentous fungal host cell comprising the expression construct according to claim 5.

12. A recombinant filamentous fungal host cell comprising the expression construct comprising the expression vector according to claim 6.


SEQUENCE LISTING (54 sequences)

SEQ ID NO: 1 - DNA, 5002 bp, Aspergillus niger
ctcgagggacaacgcatcgtttgatacacttcccgccaatatggacgttgtccagaagcctgttcagcatcgatctgggcgtctcgttctgtaagcattctcctagttactgatgactttcctctcttatctgtattccgtgaaagaggagggccactgtcctctatatagtttatggatataaaaagtttgagcttcttgccaatatgaaacagatttccccacattaagagctgtttctctataggtttccaatcaatattagtgccgtcaaaacgtttgttcagatcagattgtccacgttcgtttacagatactctgactgtagtatcatctgatctcacacgttggttgtgacgtatttttcgacgcataacattttcagcatcctgtgttatcttcgcccagtgtgaactgggtgctacagccaagtcctgttcagtgtcctttgacacagttcggttgttcagagttaccttccactcaatagtataatgaatacaaggctttcctctatgttgcctcgtagtcctttcttcgggctcctggaagaaacccagatgattgggctgggattgatgcaagggagtataaggttcatcaagtacatgttcaggtgatgggcaaaatacggatggcgtacgatctctaccgaagtcaccaggggtgggggcatacgatggagtttgtatccacggatcaggtggctgaagctgagaggcatcgtcatcgtagtaaggactaaacgtcatcccctcaaggcagtagatgccactgagaagcctagtgttgggatcatcatatgttagcctacaccatatgggtgtcccagcaagagtgtccgtgagggaagaggtgcagctaacaaaaccagtaaaatgatcaggttcatggacaatgaactaagacaggtacagtattgtagccctacccgtcttggttaacctggtaaggtcaaaaaggatcgaaccgtggctcagtacaaacaaaaggaatgttaacagtttgcgggagatgcaaggcacatgctttgtcatgtttgacgcgtttgcagtgtagaagcttccagctaccgtagattactgatacaaactcaatacactatttctataaccttactgttcaatacagtacgatcaaaatttccggaatattaatgttacggttaccttccatatgtagactagcgcacttggcattagggttcgaaatacgatcaaagagtattggggggggtgacagcagtaatgactccaactgtaaatcggcttctaggcgcgctccatctaaatgttctggctgtggtgtacaggggcataaaattacgcactacccgaatcgatagaactactcatttttatatagaagtcagaattcatggtgttttgatcattttaaatttttatatggcgggtggtgggcaactcgcttgcgcgggcaactcgcttaccgattacgttagggctgatatttacgtaaaaatcgtcaagggatgcaagaccaaagtactaaaaccccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtattgtcctgcagaatgcaatttaaactcttctgcgaatcgcttggattccccgcccctggccgtagagcttaaagtatgtcccttgtcgatgcgatgtatcacaacatataaatactagcaagggatgccatgcttggaggatagcaaccgacaacatcacatcaagctctcccttctctgaacaataaaccccacagaaggcatttatgatggtcgcgtggtggtctctatttctgtacggccttcaggtcgcggcacctgctttggctgcaacgcctgcggactggcgatcgcaatccatttatttccttctcacggatcgatttgcaaggacggatgggtcgacgactgcgacttgtaatactgcggatcaggtgtgttgttacctactagctttcagaaagaggaatgtaaactgacttgatatagaaatactgtggtggaacatggcagggcatcatcgacaaggtaaattgcccctttatcaaaaaaaaaagaaggaaaagcagaagaaaaataaaataaaaagaactctagtcctaaccatcacatagttggactatatccagggaatgggcttcacagccatctggatcacccccgttacagcccagctgccccagaccaccgcatatggagatgcctaccatggctactggcagcaggatatgtaagtcgatttctttaaatatctacctgtcatcttttacatcaatatgaactaacttgatggttttagatactctctgaacgaaaactacggcactgcagatgacttgaaggcgctctcttcggcccttcatgagagggggatgtatcttatggtcgatgtggttgctaaccatatggttcgtggtcctttgcaactgacttcgcggatatggttcatttcagtactgacaatgagtaatatcagggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccagtaagatatttctccctcattctacaacttggctgatcgatgatacttacgaaatcagttgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatgtatggttcctccaaccatgagccttcttgcaagtctcatctcctaacgaaacggctaaaaccagttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcgtaagtcttcccttttattttccgttcccaatttccacacagaaccccacctaacaagagcaaagttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaaggtaagcacaacctctaagcataccctaatggcctatcttcagagtatctgacacaagagactaatcactggcaatacagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcgtgaagggtggagagtatatgatggtactgctattcaatctggcattggacagtgagtttgagtttgatgtacataaccaaggttgtgtctgtataatatatacatgtaagatacatgagcttcggtgatataatacagaagtaccatacagtaccgcgttatgaaaacacattaatccggatcctttcctataatagactagcgtgcttggcattagggttcgaaaaacaatcgaagagtataaggggatgacagcagtaacgactccaactgtagcccacatcttgagttcggcaactactgttggcacgtgaccctgtgccttgtggtagctccttaactttgtcatcattcgaagaattttcgtcccttcccaggtaccatccaaaagacaagcatccgtcgcttcactctgagatcagatgagagtaatattgttgactgcgtttgtgatgcgggtgatgtcctctgcgatcggccgcaagctgtttagtttgccccggatcttctgtgccgacggttgctccccgaattttcttagctagtgtaatcacgctattcagaaaggcttccaagaattaggccggtagttcggcgcgtttggtgtcgtcaagctccagcagtgctggggcctcggctatgatatggttagaatgctcggggtgggtcacggcaggacacccgacactgcaacgtctaccacatttgagcgttattggcagacttgcggcgagataacgaccgctagcttgtatcaaccaaatccaactgaaattattgctttgccatcccaacagtggatttcggaggagggaggggggaagatatacgatgaacggaagactggacaagatacgttacataaagcagtactacttgtttcaaactgtgtacacaccagggctctcgcttcagcggagagtgtcgaaagattcagtaaaacatcgccaggggtgatggaaaggggttaag

SEQ ID NO: 2 - DNA, 1497 bp, Aspergillus niger
atggtcgcgtggtggtctctatttctgtacggccttcaggtcgcggcacctgctttggctgcaacgcctgcggactggcgatcgcaatccatttatttccttctcacggatcgatttgcaaggacggatgggtcgacgactgcgacttgtaatactgcggatcagaaatactgtggtggaacatggcagggcatcatcgacaagttggactatatccagggaatgggcttcacagccatctggatcacccccgttacagcccagctgccccagaccaccgcatatggagatgcctaccatggctactggcagcaggatatatactctctgaacgaaaactacggcactgcagatgacttgaaggcgctctcttcggcccttcatgagagggggatgtatcttatggtcgatgtggttgctaaccatatgggctatgatggagcgggtagctcagtcgattacagtgtgtttaaaccgttcagttcccaagactacttccacccgttctgtttcattcaaaactatgaagatcagactcaggttgaggattgctggctaggagataacactgtctccttgcctgatctcgataccaccaaggatgtggtcaagaatgaatggtacgactgggtgggatcattggtatcgaactactccattgacggcctccgtatcgacacagtaaaacacgtccagaaggacttctggcccgggtacaacaaagccgcaggcgtgtactgtatcggcgaggtgctcgacggtgatccggcctacacttgtccctaccagaacgtcatggacggcgtactgaactatcccatttactatccactcctcaacgccttcaagtcaacctccggcagcatggacgacctctacaacatgatcaacaccgtcaaatccgactgtccagactcaacactcctgggcacattcgtcgagaaccacgacaacccacggttcgcttcttacaccaacgacatagccctcgccaagaacgtcgcagcattcatcatcctcaacgacggaatccccatcatctacgccggccaagaacagcactacgccggcggaaacgaccccgcgaaccgcgaagcaacctggctctcgggctacccgaccgacagcgagctgtacaagttaattgcctccgcgaacgcaatccggaactatgccattagcaaagatacaggattcgtgacctacaagaactggcccatctacaaagacgacacaacgatcgccatgcgcaagggcacagatgggtcgcagatcgtgactatcttgtccaacaagggtgcttcgggtgattcgtataccctctccttgagtggtgcgggttacacagccggccagcaattgacggaggtcattggctgcacgaccgtgacggttggttcggatggaaatgtgcctgttcctatggcaggtgggctacctagggtattgtatccgactgagaagttggcaggtagcaagatctgtagtagctcgtga

SEQ ID NO: 3 - protein, 498 aa, Aspergillus niger
MVAWWSLFLYGLQVAAPALAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 4 - protein, 478 aa, Aspergillus niger
ATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 5 - DNA, 1710 bp, artificial sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatggtcgcgtggtggtctctatttctgtacggccttcaggtcgcggcacctgctttggctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 6 - DNA, 60 bp, Aspergillus niger
atggtcgcgtggtggtctctatttctgtacggccttcaggtcgcggcacctgctttggct

SEQ ID NO: 7 - protein, 20 aa, Aspergillus niger
MVAWWSLFLYGLQVAAPALA

SEQ ID NO: 8 - DNA, 1710 bp, Artificial sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatggttgcctggtggtccctcttcctgtacggtctccaggttgctgctcctgctcttgctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 9 - DNA, 1497 bp, Artificial sequence
  Synthetic sequence
atggttgcctggtggtccctcttcctgtacggtctccaggttgctgctcctgctcttgctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaa

SEQ ID NO: 10 - DNA, 60 bp, Artificial sequence
  Synthetic DNA
atggttgcctggtggtccctcttcctgtacggtctccaggttgctgctcctgctcttgct

SEQ ID NO: 11 - DNA, 1704 bp, Artificial sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgtccttccgctctcttctggctctgtccggtctggtctgcaccggtctggctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 12 - DNA, 1491 bp, artificial sequence
  Fusion construct
atgtccttccgctctcttctggctctgtccggtctggtctgcaccggtctggctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaa

SEQ ID NO: 13 - protein, 496 aa, Artificial Sequence
  Artificial Protein
MSFRSLLALSGLVCTGLAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 14 - DNA, 54 bp, Artificial Sequence
  Synthetic DNA
atgtccttccgctctcttctggctctgtccggtctggtctgcaccggtctggct

SEQ ID NO: 15 - protein, 18 aa, Aspergillus niger
MSFRSLLALSGLVCTGLA

SEQ ID NO: 16-20 - skipped (5 entries)

SEQ ID NO: 21 - DNA, 1701 bp, Artificial Sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatggttaagtcaattcttgcatccgttttctttgcggcgactgcgctggccgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 22 - DNA, 1488 bp, Artificial Sequence
  Fusion construct
atggttaagtcaattcttgcatccgttttctttgcggcgactgcgctggccgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaa

SEQ ID NO: 23 - protein, 495 aa, Artificial Sequence
  Fusion protein
MVKSILASVFFAATALAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 24 - DNA, 51 bp, Aspergillus niger
atggttaagtcaattcttgcatccgttttctttgcggcgactgcgctggcc

SEQ ID NO: 25 - protein, 17 aa, Aspergillus niger
MVKSILASVFFAATALA

SEQ ID NO: 26 - DNA, 1701 bp, Artificial sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatggtcaagtccatcctggcctccgtcttcttcgctgccactgctcttgctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 27 - DNA, 1488 bp, Artificial Sequence
  Fusion construct
atggtcaagtccatcctggcctccgtcttcttcgctgccactgctcttgctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaa

SEQ ID NO: 28 - protein, 495 aa, Artificial sequence; identical to SEQ ID NO: 23
  Fusion protein

SEQ ID NO: 29 - DNA, 51 bp, Artificial sequence
  Synthetic DNA
atggtcaagtccatcctggcctccgtcttcttcgctgccactgctcttgct

SEQ ID NO: 30 - DNA, 1698 bp, Artificial Sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgaagctctccgccctccttgctgccctcctggccgttgctctggctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 31 - DNA, 1485 bp, Artificial Sequence
  Fusion construct
atgaagctctccgccctccttgctgccctcctggccgttgctctggctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaa

SEQ ID NO: 32 - protein, 494 aa, Artificial Sequence
  Fusion protein
MKLSALLAALLAVALAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 33 - DNA, 48 bp, Artificial Sequence
  Synthetic DNA
atgaagctctccgccctccttgctgccctcctggccgttgctctggct

SEQ ID NO: 34 - protein, 16 aa, Artificial Sequence
  Synthetic Protein
MKLSALLAALLAVALA

SEQ ID NO: 35 - DNA, 1707 bp, Artificial sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatggtccagatcaaggttgctgctctggccatgcttttcgcctcccaggtcctctccgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 36 - DNA, 1494 bp, Artificial Sequence
  Fusion construct
atggtccagatcaaggttgctgctctggccatgcttttcgcctcccaggtcctctccgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaa

SEQ ID NO: 37 - protein, 497 aa, Artificial Sequence
  Fusion Protein
MVQIKVAALAMLFASQVLSATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 38 - DNA, 57 bp, Artificial Sequence
  Synthetic construct
atggtccagatcaaggttgctgctctggccatgcttttcgcctcccaggtcctctcc

SEQ ID NO: 39 - protein, 19 aa, Aspergillus niger
MVQIKVAALAMLFASQVLS

SEQ ID NO: 40 - DNA, 1713 bp, Artificial Sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatggtcttcaagcccctcaccatcgctgctgccattgccggtctgacccccttcgtttccgctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 41 - DNA, 1500 bp, Artificial Sequence
  Fusion construct
atggtcttcaagcccctcaccatcgctgctgccattgccggtctgacccccttcgtttccgctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaa

SEQ ID NO: 42 - protein, 499 aa, Artificial sequence
  Fusion Protein
MVFKPLTIAAAIAGLTPFVSAATPADWRSQSIYFLLTDRFARTDGSTTATCNTADQKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCFIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 43 - DNA, 63 bp, Artificial Sequence
  Synthetic construct
atggtcttcaagcccctcaccatcgctgctgccattgccggtctgacccccttcgtttccgct

SEQ ID NO: 44 - protein, 21 aa, Aspergillus niger
MVFKPLTIAAAIAGLTPFVSA

SEQ ID NO: 45 - DNA, 2123 bp, Artificial sequence
  Fusion construct
gaattcatggtgttttgatcattttaaatttttatatggcgggtggtgggcaactcgcttgcgcgggcaactcgcttaccgattacgttagggctgatatttacgtaaaaatcgtcaagggatgcaagaccaaagtactaaaaccccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtattgtcctgcagaatgcaatttaaactcttctgcgaatcgcttggattccccgcccctggccgtagagcttaaagtatgtcccttgtcgatgcgatgtatcacaacatataaatactagcaagggatgccatgcttggaggatagcaaccgacaacatcacatcaagctctcccttctctgaacaataaaccccacacaccgtcaaaatggtcgcgtggtggtctctatttctgtacggccttcaggtcgcggcacctgctttggctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 46 - DNA, 2114 bp, Artificial Sequence
  Fusion construct
gaattcatggtgttttgatcattttaaatttttatatggcgggtggtgggcaactcgcttgcgcgggcaactcgcttaccgattacgttagggctgatatttacgtaaaaatcgtcaagggatgcaagaccaaagtactaaaaccccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtattgtcctgcagaatgcaatttaaactcttctgcgaatcgcttggattccccgcccctggccgtagagcttaaagtatgtcccttgtcgatgcgatgtatcacaacatataaatactagcaagggatgccatgcttggaggatagcaaccgacaacatcacatcaagctctcccttctctgaacaataaaccccacacaccgtcaaaatggtcaagtccatcctggcctccgtcttcttcgctgccactgctcttgctgccactcctgccgactggcgctcccagtccatctacttcctgctgaccgaccgcttcgctcgtaccgatggaagcaccactgccacctgcaacactgcggaccagaagtactgcggtggtacctggcagggtatcattgacaagctcgactacatccagggcatgggtttcactgccatctggatcacccccgtgactgctcagctcccccagaccactgcctacggtgatgcctaccacggatactggcagcaggatatctactctctgaacgagaactacggcactgccgatgacctcaaggccctttcttctgctctgcacgagcgtggaatgtacctgatggtggatgttgttgccaaccacatgggctacgacggtgctggcagctctgttgactactctgtcttcaagcccttctcttcccaggactacttccaccccttctgcttcatccagaactacgaagaccagacccaggttgaggactgctggttgggtgacaacaccgtctccctccccgatcttgacaccaccaaggatgttgtcaagaacgaatggtacgactgggtgggatccctggtctccaactactccatcgatggtctccgcattgacaccgtcaagcacgtccagaaggacttctggcctggctacaacaaggctgctggtgtctactgcattggtgaggtcctcgatggagatcctgcctacacctgcccctaccagaacgtcatggatggtgttctcaactaccccatctactaccccttgctcaacgccttcaagtccacctccggcagcatggatgacctctacaacatgatcaacaccgtcaagtccgactgccccgacagcactctccttggtaccttcgtcgagaaccacgacaaccctcgtttcgccagctacaccaacgacattgctcttgccaagaacgtcgctgctttcatcatcctgaacgacggtatccccatcatctacgctggccaggagcagcactacgctggtggcaacgaccctgccaaccgtgaggccacctggctgtctggctaccccaccgacagcgaattgtacaagttgattgcctctgccaacgccatccgcaactacgccatctccaaggacactggtttcgtcacctacaagaactggcccatctacaaggatgacaccaccattgccatgcgcaagggtactgatggcagccagatcgtcaccatcctgtccaacaagggtgcctccggtgactcctacaccctctccctctccggtgctggctacactgctggccagcagctgaccgaggtcattggctgcaccaccgtcaccgttggatcggatggcaacgtgcctgtgcccatggccggtggtcttcctcgtgtcctctaccccactgagaagcttgctggcagcaagatctgctcgtcgtcgtaaattaattaa

SEQ ID NO: 47 - DNA, 2028 bp, Artificial Sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgaagtccactattatcacctccattctcttctctgtggctgccgtccaggcctactctcctgctgagcagatcgatgtccagtcccacctcctctccgaccccaccaaggttgagggtgagacctacgactacgtcattgctggtggtggtctgaccggtctgaccgttgctgccaagctctccgagaaccccaagatcaaggtcctggtcattgagaagggtttctacgagagcaacgacggccccatcattgaggaccccaacgcctacggcgagatcttcggtacctccgttgaccagaactacctgaccgttcccttgatcaacaaccgcactggtgagatcaagtccggtcttggtcttggtggcagcactctgatcaacggtgacagctggactcgccccgacaaggtccagattgacagctgggagaaggtcttcggtatggaaggctggaactgggacaacgtcttccagtacatgcagaaggctgagcgctcccgccctcctactgctgctcagattgaggctggccacttctacgaccccgcctgccacggtaccgacggcaccgtccacgctggtcctcgtgacaacggcaagccctggagccctctgatgcgtgctctgatgaacaccgtctctgctttcggtgttcctgtccagaaggacttccactgcggtcacccccgtggtgtctccatgatccccaacaacctgcacgagaaccagatccgtgccgatgctgcccgtgagtggctactccccaactaccagcgtgacaacctgcagatcctgaccggccagaaggtcggcaaggtcctcttcaaccagactgcctccggtcccaaggccgttggtgtcaacttcggtaccaacaaggccgtcaacttcaacgtctacgccaagcaggaagtcctccttgctgctggctctgccatctcccctctgatcctggaatactctggtatcggtatcaagtccgtcctcgacaaggccggtgtcaagcagctcctcgagcttcctgtcggtctcaacatgcaggaccagaccaccaccaccgtccgctcccgtgccaacaacgcccccggccagggccaggctgcctacttcgccaacttcaccgaggtccttggtgaccacgctgcccagggtatcaagctcctcgacaccaagctcgaccagtgggctgaggagactgttgctcgtggtggtttccacaacgtcactgctctcaagatccagtacgagaactaccgcaactggctactcgatgaggatgttgctttcgccgagctcttcttcgacaccgagggcaagatcaacttcgacatctggaacctgatccccttcacccgtggctccgtccacatcctgtcttccgacccctacctctggcagtacgccaacgaccccaagttcttcatgaacgagcttgacctccttggccaggctgctgccaccaagctcggtcgtgagctttcttctgctggtgagatgaagaagtactacgccggtgagaccatccccggtgacaacctgccccaggatgccactgttgagcagtgggaagactacgtcatgatgaacttccgccccaactggcacgccgtctccacctgctccatgatgagccgtgagcttggtggtgttgttgatgccactgccaaggtctacggcacccagggtctccgtgtcattgatggcagcattcctcctactcaggtctcctcccacgtcatgactgtcttctacggcatggccctccgcattgctgagtccgtcctcgaggactacgccaagaaggcttaaattaattaa

SEQ ID NO: 48 - DNA, 1815 bp, Artificial Sequence
  Fusion construct
atgaagtccactattatcacctccattctcttctctgtggctgccgtccaggcctactctcctgctgagcagatcgatgtccagtcccacctcctctccgaccccaccaaggttgagggtgagacctacgactacgtcattgctggtggtggtctgaccggtctgaccgttgctgccaagctctccgagaaccccaagatcaaggtcctggtcattgagaagggtttctacgagagcaacgacggccccatcattgaggaccccaacgcctacggcgagatcttcggtacctccgttgaccagaactacctgaccgttcccttgatcaacaaccgcactggtgagatcaagtccggtcttggtcttggtggcagcactctgatcaacggtgacagctggactcgccccgacaaggtccagattgacagctgggagaaggtcttcggtatggaaggctggaactgggacaacgtcttccagtacatgcagaaggctgagcgctcccgccctcctactgctgctcagattgaggctggccacttctacgaccccgcctgccacggtaccgacggcaccgtccacgctggtcctcgtgacaacggcaagccctggagccctctgatgcgtgctctgatgaacaccgtctctgctttcggtgttcctgtccagaaggacttccactgcggtcacccccgtggtgtctccatgatccccaacaacctgcacgagaaccagatccgtgccgatgctgcccgtgagtggctactccccaactaccagcgtgacaacctgcagatcctgaccggccagaaggtcggcaaggtcctcttcaaccagactgcctccggtcccaaggccgttggtgtcaacttcggtaccaacaaggccgtcaacttcaacgtctacgccaagcaggaagtcctccttgctgctggctctgccatctcccctctgatcctggaatactctggtatcggtatcaagtccgtcctcgacaaggccggtgtcaagcagctcctcgagcttcctgtcggtctcaacatgcaggaccagaccaccaccaccgtccgctcccgtgccaacaacgcccccggccagggccaggctgcctacttcgccaacttcaccgaggtccttggtgaccacgctgcccagggtatcaagctcctcgacaccaagctcgaccagtgggctgaggagactgttgctcgtggtggtttccacaacgtcactgctctcaagatccagtacgagaactaccgcaactggctactcgatgaggatgttgctttcgccgagctcttcttcgacaccgagggcaagatcaacttcgacatctggaacctgatccccttcacccgtggctccgtccacatcctgtcttccgacccctacctctggcagtacgccaacgaccccaagttcttcatgaacgagcttgacctccttggccaggctgctgccaccaagctcggtcgtgagctttcttctgctggtgagatgaagaagtactacgccggtgagaccatccccggtgacaacctgccccaggatgccactgttgagcagtgggaagactacgtcatgatgaacttccgccccaactggcacgccgtctccacctgctccatgatgagccgtgagcttggtggtgttgttgatgccactgccaaggtctacggcacccagggtctccgtgtcattgatggcagcattcctcctactcaggtctcctcccacgtcatgactgtcttctacggcatggccctccgcattgctgagtccgtcctcgaggactacgccaagaaggcttaa

SEQ ID NO: 49 - protein, 604 aa, Artificial Sequence
  Fusion Protein
MKSTIITSILFSVAAVQAYSPAEQIDVQSHLLSDPTKVEGETYDYVIAGGGLTGLTVAAKLSENPKIKVLVIEKGFYESNDGPIIEDPNAYGEIFGTSVDQNYLTVPLINNRTGEIKSGLGLGGSTLINGDSWTRPDKVQIDSWEKVFGMEGWNWDNVFQYMQKAERSRPPTAAQIEAGHFYDPACHGTDGTVHAGPRDNGKPWSPLMRALMNTVSAFGVPVQKDFHCGHPRGVSMIPNNLHENQIRADAAREWLLPNYQRDNLQILTGQKVGKVLFNQTASGPKAVGVNFGTNKAVNFNVYAKQEVLLAAGSAISPLILEYSGIGIKSVLDKAGVKQLLELPVGLNMQDQTTTTVRSRANNAPGQGQAAYFANFTEVLGDHAAQGIKLLDTKLDQWAEETVARGGFHNVTALKIQYENYRNWLLDEDVAFAELFFDTEGKINFDIWNLIPFTRGSVHILSSDPYLWQYANDPKFFMNELDLLGQAAATKLGRELSSAGEMKKYYAGETIPGDNLPQDATVEQWEDYVMMNFRPNWHAVSTCSMMSRELGGVVDATAKVYGTQGLRVIDGSIPPTQVSSHVMTVFYGMALRIAESVLEDYAKKA

SEQ ID NO: 50 - DNA, 54 bp, Aspergillus niger
atgaagtccactattatcacctccattctcttctctgtggctgccgtccaggcc

SEQ ID NO: 51 - protein, 18 aa, Aspergillus niger
MKSTIITSILFSVAAVQA

SEQ ID NO: 52 - DNA, 2025 bp, Artificial Sequence
  Fusion construct
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatggtcaagtccatcctggcctccgtcttcttcgctgccactgctcttgcttactctcctgctgagcagatcgatgtccagtcccacctcctctccgaccccaccaaggttgagggtgagacctacgactacgtcattgctggtggtggtctgaccggtctgaccgttgctgccaagctctccgagaaccccaagatcaaggtcctggtcattgagaagggtttctacgagagcaacgacggccccatcattgaggaccccaacgcctacggcgagatcttcggtacctccgttgaccagaactacctgaccgttcccttgatcaacaaccgcactggtgagatcaagtccggtcttggtcttggtggcagcactctgatcaacggtgacagctggactcgccccgacaaggtccagattgacagctgggagaaggtcttcggtatggaaggctggaactgggacaacgtcttccagtacatgcagaaggctgagcgctcccgccctcctactgctgctcagattgaggctggccacttctacgaccccgcctgccacggtaccgacggcaccgtccacgctggtcctcgtgacaacggcaagccctggagccctctgatgcgtgctctgatgaacaccgtctctgctttcggtgttcctgtccagaaggacttccactgcggtcacccccgtggtgtctccatgatccccaacaacctgcacgagaaccagatccgtgccgatgctgcccgtgagtggctactccccaactaccagcgtgacaacctgcagatcctgaccggccagaaggtcggcaaggtcctcttcaaccagactgcctccggtcccaaggccgttggtgtcaacttcggtaccaacaaggccgtcaacttcaacgtctacgccaagcaggaagtcctccttgctgctggctctgccatctcccctctgatcctggaatactctggtatcggtatcaagtccgtcctcgacaaggccggtgtcaagcagctcctcgagcttcctgtcggtctcaacatgcaggaccagaccaccaccaccgtccgctcccgtgccaacaacgcccccggccagggccaggctgcctacttcgccaacttcaccgaggtccttggtgaccacgctgcccagggtatcaagctcctcgacaccaagctcgaccagtgggctgaggagactgttgctcgtggtggtttccacaacgtcactgctctcaagatccagtacgagaactaccgcaactggctactcgatgaggatgttgctttcgccgagctcttcttcgacaccgagggcaagatcaacttcgacatctggaacctgatccccttcacccgtggctccgtccacatcctgtcttccgacccctacctctggcagtacgccaacgaccccaagttcttcatgaacgagcttgacctccttggccaggctgctgccaccaagctcggtcgtgagctttcttctgctggtgagatgaagaagtactacgccggtgagaccatccccggtgacaacctgccccaggatgccactgttgagcagtgggaagactacgtcatgatgaacttccgccccaactggcacgccgtctccacctgctccatgatgagccgtgagcttggtggtgttgttgatgccactgccaaggtctacggcacccagggtctccgtgtcattgatggcagcattcctcctactcaggtctcctcccacgtcatgactgtcttctacggcatggccctccgcattgctgagtccgtcctcgaggactacgccaagaaggcttaaattaattaa

SEQ ID NO: 53 - DNA, 1812 bp, Artificial Sequence
  Fusion Construct
atggtcaagtccatcctggcctccgtcttcttcgctgccactgctcttgcttactctcctgctgagcagatcgatgtccagtcccacctcctctccgaccccaccaaggttgagggtgagacctacgactacgtcattgctggtggtggtctgaccggtctgaccgttgctgccaagctctccgagaaccccaagatcaaggtcctggtcattgagaagggtttctacgagagcaacgacggccccatcattgaggaccccaacgcctacggcgagatcttcggtacctccgttgaccagaactacctgaccgttcccttgatcaacaaccgcactggtgagatcaagtccggtcttggtcttggtggcagcactctgatcaacggtgacagctggactcgccccgacaaggtccagattgacagctgggagaaggtcttcggtatggaaggctggaactgggacaacgtcttccagtacatgcagaaggctgagcgctcccgccctcctactgctgctcagattgaggctggccacttctacgaccccgcctgccacggtaccgacggcaccgtccacgctggtcctcgtgacaacggcaagccctggagccctctgatgcgtgctctgatgaacaccgtctctgctttcggtgttcctgtccagaaggacttccactgcggtcacccccgtggtgtctccatgatccccaacaacctgcacgagaaccagatccgtgccgatgctgcccgtgagtggctactccccaactaccagcgtgacaacctgcagatcctgaccggccagaaggtcggcaaggtcctcttcaaccagactgcctccggtcccaaggccgttggtgtcaacttcggtaccaacaaggccgtcaacttcaacgtctacgccaagcaggaagtcctccttgctgctggctctgccatctcccctctgatcctggaatactctggtatcggtatcaagtccgtcctcgacaaggccggtgtcaagcagctcctcgagcttcctgtcggtctcaacatgcaggaccagaccaccaccaccgtccgctcccgtgccaacaacgcccccggccagggccaggctgcctacttcgccaacttcaccgaggtccttggtgaccacgctgcccagggtatcaagctcctcgacaccaagctcgaccagtgggctgaggagactgttgctcgtggtggtttccacaacgtcactgctctcaagatccagtacgagaactaccgcaactggctactcgatgaggatgttgctttcgccgagctcttcttcgacaccgagggcaagatcaacttcgacatctggaacctgatccccttcacccgtggctccgtccacatcctgtcttccgacccctacctctggcagtacgccaacgaccccaagttcttcatgaacgagcttgacctccttggccaggctgctgccaccaagctcggtcgtgagctttcttctgctggtgagatgaagaagtactacgccggtgagaccatccccggtgacaacctgccccaggatgccactgttgagcagtgggaagactacgtcatgatgaacttccgccccaactggcacgccgtctccacctgctccatgatgagccgtgagcttggtggtgttgttgatgccactgccaaggtctacggcacccagggtctccgtgtcattgatggcagcattcctcctactcaggtctcctcccacgtcatgactgtcttctacggcatggccctccgcattgctgagtccgtcctcgaggactacgccaagaaggcttaa

SEQ ID NO: 54 - protein, 603 aa, Artificial Sequence
  Fusion construct
MVKSILASVFFAATALAYSPAEQIDVQSHLLSDPTKVEGETYDYVIAGGGLTGLTVAAKLSENPKIKVLVIEKGFYESNDGPIIEDPNAYGEIFGTSVDQNYLTVPLINNRTGEIKSGLGLGGSTLINGDSWTRPDKVQIDSWEKVFGMEGWNWDNVFQYMQKAERSRPPTAAQIEAGHFYDPACHGTDGTVHAGPRDNGKPWSPLMRALMNTVSAFGVPVQKDFHCGHPRGVSMIPNNLHENQIRADAAREWLLPNYQRDNLQILTGQKVGKVLFNQTASGPKAVGVNFGTNKAVNFNVYAKQEVLLAAGSAISPLILEYSGIGIKSVLDKAGVKQLLELPVGLNMQDQTTTTVRSRANNAPGQGQAAYFANFTEVLGDHAAQGIKLLDTKLDQWAEETVARGGFHNVTALKIQYENYRNWLLDEDVAFAELFFDTEGKINFDIWNLIPFTRGSVHILSSDPYLWQYANDPKFFMNELDLLGQAAATKLGRELSSAGEMKKYYAGETIPGDNLPQDATVEQWEDYVMMNFRPNWHAVSTCSMMSRELGGVVDATAKVYGTQGLRVIDGSIPPTQVSSHVMTVFYGMALRIAESVLEDYAKKA